(19) United States
(12) Patent Application Publication
(10) Pub. No.: US 2022/0214195 A1
(43) Pub. Date: Jul. 7, 2022

(54) FLOW RATE MEASURING DEVICE

(71) Applicant: FUJIFILM Corporation, Tokyo (JP)

(72) Inventor: Takehiro KASAHARA, Minamiashigara-shi (JP)

(73) Assignee: FUJIFILM Corporation, Tokyo (JP)

(21) Appl. No.: 17/704,371

(22) Filed: Mar. 25, 2022

Related U.S. Application Data
(63) Continuation of application No. PCT/JP2020/035999, filed on Sep. 24, 2020.

(30) Foreign Application Priority Data
Sep. 27, 2019 (JP) 2019-177825

(51) Int. Cl. G01F 1/661; G01F 1/663

(52) U.S. Cl. CPC G01F 1/661; A61B 5/02108; G01F 1/663

(57) ABSTRACT

Provided is a flow rate measuring device capable of ensuring sufficient signal intensity even with a low output light source. The flow rate measuring device that detects a flow velocity of a fluid flowing through an object by a Doppler effect of light, the flow rate measuring device includes a light source unit that emits laser light to the object, a light receiving unit that receives the laser light scattered by the object, and a light bending member that bends the laser light emitted from the light source unit and causes the laser light to be inclined with respect to a surface of the object and incident on the surface.

### CROSS-REFERENCE TO RELATED APPLICATIONS

This application is a Continuation of PCT International Application No. PCT/JP2020/035999 filed on Sep. 24, 2020, which claims priority under 35 U.S.C. § 119(a) to Japanese Patent Application No. 2019-177825 filed on Sep. 27, 2019. The above application is hereby expressly incorporated by reference, in its entirety, into the present application.

### BACKGROUND OF THE INVENTION

### 1. Field of the Invention

The present invention relates to a flow rate measuring device.

### 2. Description of the Related Art

There has been known a device that irradiates a living body with light and that detects the light reflected by the living body to acquire information regarding the living body, such as a blood flow velocity and a pulse wave. Further, there have been proposed a method of converting a pulse wave velocity by heartbeat measurement at a chest and pulse measurement at a fingertip into a blood pressure, and a method of converting a blood flow velocity into a blood pressure.

Incidentally, it has been considered that depression is caused by chronic stress. It has been known that chronic stress correlates with long-term fluctuations in blood pressure, and if a blood pressure can be measured continuously, it is considered that chronic stress levels can be measured. In that regard, a wearable measuring device that can measure a blood pressure continuously by measuring a blood flow velocity or a pulse wave with a measuring device which is always worn has been proposed.

For example, JP2017-094173A discloses an information detector that detects information regarding a measurement target, the information detector comprising: an irradiation unit that emits light; a reflection unit having a reflectivity different from the measurement target; a light receiving unit that receives return light of the light emitted from the irradiation unit; a determination unit that determines that a measurement error has occurred in a case where an amount of light received in the light receiving unit is larger than a first threshold value to output measurement error alarm information and that determines that an abnormality has occurred in the irradiation unit or the light receiving unit in a case where the amount of light received in the light receiving unit is smaller than a second threshold value.

JP2017-094173A discloses that a measuring device is worn on a fingertip of a living body to acquire information regarding the living body.

### SUMMARY OF THE INVENTION

In the case of the method of converting a pulse wave velocity by heartbeat measurement at the chest and pulse measurement at the fingertip into a blood pressure, it is necessary to wear a measuring device on the fingertip, and there is reluctance to wear the measuring device. Therefore, the inventors have considered realizing continuous measurement of a blood pressure with a wristband-type measuring device that does not give a feeling of reluctance to wear the measuring device.

As a medical device, there is blood flow velocity measurement (laser Doppler method) using a near-infrared laser, and it has been known that there is a high correlation between the blood flow velocity of a radial artery (wrist) and a blood pressure.

For this reason, the inventors have considered that a blood pressure can be continuously measured if blood flow velocity measurement can be realized with a wristband-type measuring device worn on the wrist.

However, according to the study by the inventors, in the case of the wearable measuring device, it was found that there is a problem that the reflected light from the living body cannot be detected with the small irradiation intensity of laser light, although it is difficult to increase the laser output (irradiation intensity) for downsizing and power saving of the device.

An object of the present invention is to solve such a problem of the related art, and to provide a flow rate measuring device capable of ensuring sufficient signal intensity even with a low output light source.

In order to achieve the object, the present invention has the following configurations.

[1] A flow rate measuring device that detects a flow velocity of a fluid flowing through an object by a Doppler effect of light, the flow rate measuring device comprising:

a light source unit that emits laser light to the object;

a light receiving unit that receives the laser light scattered by the object; and

a light bending member that bends the laser light emitted from the light source unit and causes the laser light to be inclined with respect to a surface of the object and incident on the surface.

[2] The flow rate measuring device according to [1], further comprising:

a holding mechanism that keeps a distance between the light bending member and the object constant.

[3] The flow rate measuring device according to [1] or [2], in which the laser light bent by the light bending member is incident on the object at an angle of 30° to 70° with respect to a perpendicular line to the surface of the object.

[4] The flow rate measuring device according to any one of [1] to (3), in which the light bending member includes at least one of a prism sheet, a lens sheet, or a liquid crystal diffraction element.

[5] The flow rate measuring device according to any one of [1] to [4], in which the light source unit includes a substrate and a plurality of laser oscillating elements provided on the substrate.

[6] The flow rate measuring device according to any one of [1] to [4], in which the light source unit has

a laser oscillating element,

an alignment mechanism that performs scanning with laser light emitted from the laser oscillating element, and

an optical member that makes an incidence angle of the laser light with which the scanning is performed by the alignment mechanism, on the light bending member constant.

[7] The flow rate measuring device according to [6], in which the optical member includes any one of a liquid crystal lens or a gradient index lens.

According to the present invention, it is possible to provide a flow rate measuring device capable of ensuring sufficient signal intensity even with a low output light source.

### BRIEF DESCRIPTION OF THE DRAWINGS

FIG. 1 is a view schematically showing an example of a flow rate measuring device of an embodiment of the present invention.

FIG. 2 is a schematic view illustrating flow rate measurement.

FIG. 3 is a view illustrating an incidence angle of laser in a case of the flow rate measurement.

FIG. 4 is a view illustrating an incidence angle of laser in a case of the flow rate measurement.

FIG. 5 is a graph showing a relationship between a distance and a signal intensity, for showing a difference in signal intensity based on an incidence angle of laser.

FIG. 6 is a view schematically showing an example of a prism sheet that is used as a light bending member.

FIG. 7 is a view schematically showing an example of a diffraction element that is used as the light bending member.

FIG. 8 is a view schematically showing an example of a liquid crystal diffraction element that is used as the light bending member.

FIG. 9 is a plan view showing the liquid crystal diffraction element shown in FIG. 8.

FIG. 10 is a view schematically showing an example of an exposure device that exposes an alignment film of the liquid crystal diffraction element shown in FIG. 8.

FIG. 11 is a conceptual view illustrating an action of the liquid crystal diffraction element shown in FIG. 8.

FIG. 12 is a conceptual view illustrating the action of the liquid crystal diffraction element shown in FIG. 8.

FIG. 13 is a view schematically showing an example of the liquid crystal diffraction element.

FIG. 14 is a view schematically showing an example of a prism that is used as the light bending member.

FIG. 15 is a view schematically showing an example of a mirror that is used as the light bending member.

FIG. 16 is a cross-sectional view schematically showing an example in which a user wears the flow rate measuring device of the embodiment of the present invention.

FIG. 17 is a side view of a part of the flow rate measuring device shown in FIG. 16.

FIG. 18 is a perspective view of FIG. 17.

FIG. 19 is a perspective view schematically showing another example of the flow rate measuring device of the embodiment of the present invention.

FIG. 20 is a plan view schematically showing a light source unit of FIG. 19.

FIG. 21 is a perspective view schematically showing another example of the flow rate measuring device of the embodiment of the present invention.

FIG. 22 is a view schematically showing the light source unit of FIG. 21.

FIG. 23 is a view schematically showing an example of a second liquid crystal layer included in an optical member shown in FIG. 21.

FIG. 24 is a plan view of the second liquid crystal layer shown in FIG. 23.

FIG. 25 is a view schematically showing an example of an exposure device that exposes an alignment film which forms the second liquid crystal layer shown in FIG. 23.

FIG. 26 is a view schematically showing an example of a laminate including the liquid crystal diffraction element.

### DESCRIPTION OF THE PREFERRED EMBODIMENTS

Hereinafter, a flow rate measuring device of an embodiment of the present invention will be described in detail on the basis of suitable embodiments shown in the attached drawings.

In the present specification, the numerical range represented by “to” means a range including the numerical values before and after “to” as the lower limit value and the upper limit value.

[Flow Rate Measuring Device]

A flow rate measuring device of the embodiment of the present invention is a flow rate measuring device that detects a flow velocity of a fluid flowing through an object by a Doppler effect of light, the flow rate measuring device comprising:

a light source unit that emits laser light to the object:

a light receiving unit that receives the laser light scattered by the object; and

a light bending member that bends the laser light emitted from the light source unit and that causes the laser light to be inclined with respect to a surface of the object and incident on the surface.

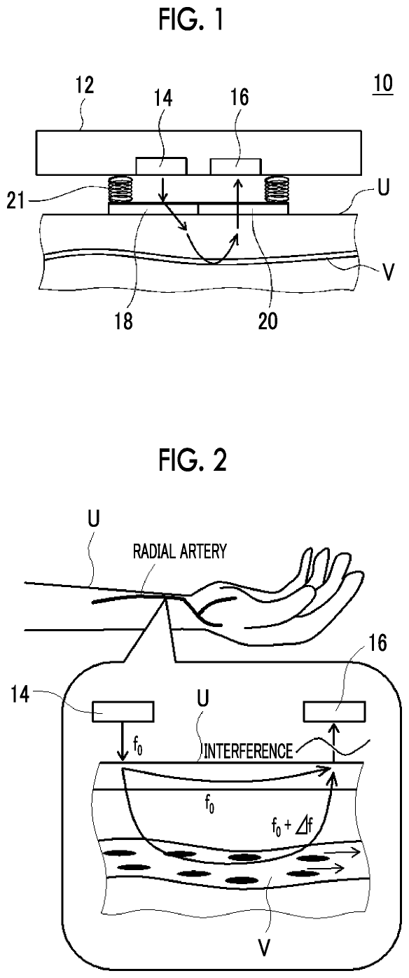

FIG. 1 conceptually shows an example of the flow rate measuring device of the embodiment of the present invention.

A flow rate measuring device 10 shown in FIG. 1 is a device that irradiates a living body with light and that detects light reflected by the living body to acquire information regarding a blood flow velocity.

The flow rate measuring device 10 shown in FIG. 1 includes a substrate 12, a laser light source 14, a light receiving unit 16, a light bending member 18, a light condensing member 20, and a holding mechanism 21. The laser light source 14 is a light source unit in the present invention. The flow rate measuring device of the embodiment of the present invention preferably includes the light condensing member 20 and the holding mechanism 21.

As shown in FIG. 1, the laser light source 14 and the light receiving unit 16 are disposed on the substrate 12 so as to be spaced apart from each other by a predetermined distance in a plane direction of a surface of the substrate 12. The light bending member 18 is disposed so as to face the laser light source 14 in a direction perpendicular to the surface of the substrate 12. Further, the light condensing member 20 is disposed so as to face the light receiving unit 16 in the direction perpendicular to the surface of the substrate 12. The light bending member 18 and the light condensing member 20 are fixed to the substrate 12 via a holding mechanism 21.

As shown in FIG. 1, the flow rate measuring device 10 is worn on the wrist of a user U (object) such that the light bending member 18 is on the user U side. At that time, the flow rate measuring device 10 is worn such that the laser light source 14 is disposed on the upstream side in a flow direction of a blood vessel (radial artery) V of the wrist of the user U and the light receiving unit 16 is disposed on the downstream side. Further, the light bending member 18 and the light condensing member 20 are worn so as to be in contact with the user U.

In the flow rate measuring device 10 worn on the wrist of the user U, laser light is emitted from the laser light source 14, the emitted laser light is bent by the light bending member 18 and incident on the user U, the light scattered and reflected in the body is condensed by the light condensing member 20 in a direction of the light receiving unit 16, and the light is received by the light receiving unit 16. The flow rate measuring device 10 measures the blood flow velocity by using the so-called laser Doppler method from the result in which the light is received by the light receiving unit 16.

Here, the laser Doppler method will be described with reference to FIG. 2.

In FIG. 2, the laser light source 14 is disposed on the upstream side in the flow direction of the blood vessel (radial artery) V of the wrist of the user U, and the light receiving unit 16 is disposed on the downstream side.

In a case where the user U is irradiated with laser light having a frequency f₀ from the laser light source 14, the light receiving unit 16 receives a component of the laser light that propagates near the epidermis of the user U and a component of the laser light that is reflected by hemoglobin in the blood vessel V to propagate.

The frequency of the laser light propagating near the epidermis remains f₀.

Meanwhile, the frequency of the laser light reflected by the hemoglobin in the blood vessel V is changed to f₀+Δf according to the moving speed of the hemoglobin. Therefore, fast Fourier transform (FFT) is performed on the frequency data of the light received by the light receiving unit 16 to obtain the frequency of the laser light reflected by the hemoglobin in the blood vessel V, so that the blood flow velocity can be calculated from the change from the frequency f₀ of the emitted laser light.

As a method for calculating the blood flow velocity by the laser Doppler method, a conventionally well-known method may be used. Examples of the method include the method described in JP2012-210321 A and the method described in JP2017-192629A.

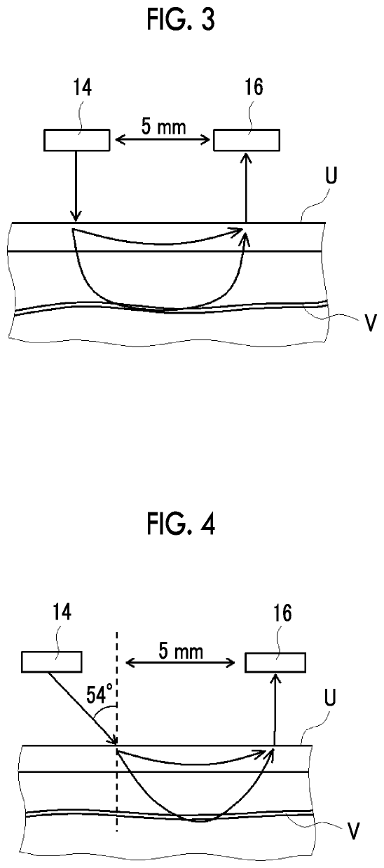

Here, according to the study by the inventors, as shown in FIG. 3, it was found that there is a problem that the intensity of laser light received by the light receiving unit 16 is low and the laser light cannot be sufficiently detected, in a case where the laser light emitted by the laser light source 14 is incident from a direction substantially perpendicular to the surface of the user U.

On the other hand, as shown in FIG. 4, in a case where the laser light emitted by the laser light source 14 is incident from an oblique direction with respect to the surface of the user U such that the traveling direction is directed to the light receiving unit 16 side, the intensity of the laser light received by the light receiving unit 16 becomes high and the laser light can be sufficiently detected.

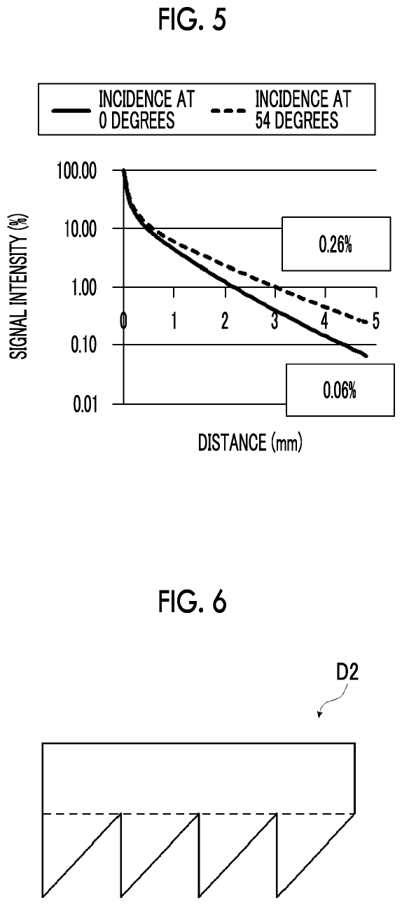

FIG. 5 shows a graph showing the relationship between the distance (mm) and the signal intensity (%) in a case where the angle of the incidence light with respect to the perpendicular line to the surface of the user U is 0° and 54°. In FIG. 5, the distance on the horizontal axis is a distance from the point where the laser light is incident on the user U to the point where the laser light is emitted. Generally, in the laser Doppler method, the distance from the incidence point to the emission point is measured as a distance of about twice the depth of the blood vessel for which blood flow is measured. Since the depth of the blood vessel (radial artery) of the wrist is about 2 mm to 3 mm, the distance from the incidence point to the emission point is about 5 mm. Further, in FIG. 5, the signal intensity on the vertical axis is a ratio of the intensity of the received laser light to the intensity of the incident laser light.

As shown in FIG. 5, the intensity of light received at a distance of 5 mm is increased by four times or more in a case where the angle of the incidence light is 54°, as compared with the case where the angle is 0°.

As described above, in the case of the wearable measuring device, there is a problem that the reflected light from the living body cannot be sufficiently detected with the small irradiation intensity of laser light because the intensity of light received is low, although it is necessary to reduce the irradiation intensity of laser light because it is difficult to increase the laser output (irradiation intensity) for downsizing and power saving of the device.

In response to this, as described above, the intensity of light received can be improved with laser light incident from the oblique direction with respect to the surface of the user U.

Here, it is also conceivable to tilt the laser light source itself so that the laser light is incident from the oblique direction with respect to the surface of the user U. Generally, the laser light source has a configuration in which the laser light is emitted in a direction parallel or perpendicular to the substrate on which the laser light source is provided. Therefore, in a case where the laser light source itself is tilted, it is necessary to tilt the entire substrate. However, it is not easy to tilt the entire substrate in a case where the measuring device is made wearable.

In response to this, the flow rate measuring device 10 of the embodiment of the present invention has the light bending member 18 that bends laser light emitted from the laser light source 14 and that causes the laser light to be incident from a direction inclined with respect to the surface of the user U.

With such a configuration, the laser light can be incident from the oblique direction with respect to the surface of the user U, so that the intensity of light received can be improved even in a case where the irradiation intensity of the laser light source 14 is low.

Further, with the configuration in which the light bending member 18 is used, the measuring device can be easily made wearable because it is not necessary to tilt the laser light source together with the substrate.

The incidence angle of the laser light bent by the light bending member 18 on the user U is preferably 30° to 70°, more preferably 40° to 60°, and still more preferably 50° to 55°, with respect to the perpendicular line to the surface of the user U.

<Substrate>

The substrate 12 is a substrate on which the laser light source 14 and the light receiving unit 16 are mounted. The substrate 12 is not particularly limited, and a semiconductor substrate that is used as a substrate on which the laser light source 14 and/or the light receiving unit 16 is mounted may be appropriately used.

In the example shown in FIG. 1, the laser light source 14 and the light receiving unit 16 are mounted on the substrate 12, but the present invention is not limited thereto, and in the substrate 12, a substrate on which the laser light source 14 is mounted and a substrate on which the light receiving unit 16 is mounted may be separate substrates.

Further, for example, an integrated circuit that calculates the blood flow velocity by using the laser Doppler method from a photodetection signal output by the light receiving unit 16, or further calculates the blood pressure from the blood flow velocity may be mounted on the substrate 12.

The correlation between a blood flow velocity and a blood pressure has been known and can be obtained, for example, from the relationship described in JP2013-132437A.

<Laser Light Source>

The laser light source 14 is used to irradiate the user U with laser light. The laser light source 14 need only be a laser light source that emits laser light having a wavelength which is used to measure the blood flow velocity by the laser Doppler method. The laser light emitted by the laser light source 14 is preferably near-infrared light (a wavelength of 650 nm to 1400 nm).

The laser light source 14 may be an end surface emitting laser that emits light in a direction parallel to the substrate 12, but is preferably a surface emitting laser that emits light in a direction perpendicular to the substrate 12.

The lower limit of the intensity of the light emitted by the laser light source 14 is preferably 0.3 mW or more, more preferably 0.4 mW or more, and still more preferably 0.5 mW or more, from the viewpoint of ensuring the intensity of light received and the like. Further, the upper limit is preferably 2 mW or less, more preferably 0.6 mW or less, and still more preferably 0.4 mW, from the viewpoint of downsizing, power saving, or the like of the device.

<Light Receiving Unit>

The light receiving unit 16 receives (detects) laser light reflected in the body of the user U. The light receiving unit 16 is mounted on the substrate 12 with the light receiving surface thereof facing a direction perpendicular to the substrate 12.

As the light receiving unit 16, a photodetection device that is used to measure the blood flow velocity by the laser Doppler method may be used. For example, the light receiving unit 16 includes a photoelectric conversion element such as a photodiode, which outputs an electric current according to the amount of received light, an amplification circuit that amplifies the output electric current of the photoelectric conversion element, an electric current/voltage conversion circuit that converts an electric current signal into a voltage signal, and the like.

The light receiving unit 16 converts the received light into a voltage signal and outputs the voltage signal as a photodetection signal.

The size of the light receiving unit 16 is not limited as long as the light receiving unit 16 can receive (detect) the laser light reflected in the body of the user U, but it is preferable that the area thereof is enlarged so that the capture angle is increased, in terms of obtaining high detection sensitivity.

<Light Bending Member>

The light bending member 18 is a member that bends the laser light emitted from the laser light source 14 and that causes the laser light to be inclined with respect to the surface of the user U and incident on the surface. The light bending member 18 is disposed between the laser light source 14 and the user U in a direction in which the laser light of the laser light source 14 is emitted.

As the light bending member 18, a prism sheet, a diffraction element, a lens sheet, a liquid crystal diffraction element, a prism, a mirror, or the like may be used.

(Prism Sheet)

The prism sheet is a prism sheet having a predetermined refractive index and having a fine uneven shape in which a plurality of unit prisms are arranged on a surface thereof, on a transparent base material, and refracts light to bend laser light. As the prism sheet that is used as the light bending member 18, a conventionally well-known prism sheet may be appropriately used as long as laser light can be bent at a desired angle.

FIG. 6 shows a schematic view of an example of the prism sheet that is used as the light bending member 18. A prism sheet D2 shown in FIG. 6 has a structure in which a plurality of unit prisms having a right angled triangular cross section are arranged.

The period, material (refractive index), height of the prism, and the like of the prism structure in the prism sheet need only be appropriately set according to the wavelength of laser light to be refracted, the angle of refraction, and the like.

(Diffraction Element)

The diffraction element has a configuration in which fine linear grooves and slits are alternately arranged on a surface of a film-like material in parallel at a predetermined period, and bends laser light by diffraction. As the diffraction element that is used as the light bending member 18, a conventionally well-known diffraction element may be appropriately used as long as laser light can be bent at a desired angle.

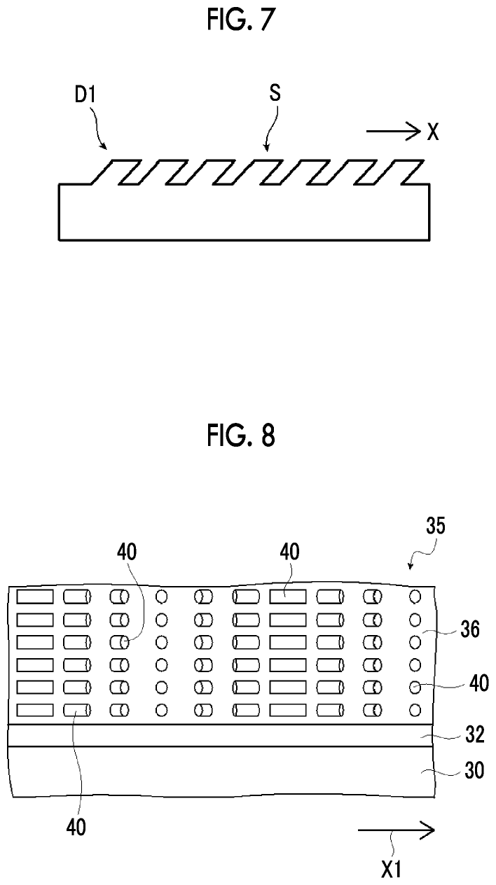

FIG. 7 shows a schematic view of an example of a diffraction element that is used as the light bending member 18. A diffraction element D1 shown in FIG. 7 has a configuration in which fine linear grooves and slits are alternately arranged on the surface thereof in parallel at a predetermined period.

The period, material, heights of groove and slit, and the like of the uneven structure in the diffraction element need only be appropriately set according to the wavelength of laser light to be diffracted, the angle of diffraction, and the like.

(Lens Sheet)

The lens sheet is, for example, a Fresnel lens sheet having a plurality of lens surfaces arranged along a certain arrangement direction, in which the inclination angles of the lens surfaces with respect to the sheet surface are gradually changed along the arrangement direction. In a typical linear Fresnel lens sheet, the inclination angles of the lens surfaces with respect to the sheet surface are increased in order from the center side to the outside in the arrangement direction of the lens surfaces.

The period, inclination angle, material (refractive index), and the like of the lens surface in the Fresnel lens sheet need only be appropriately set according to the wavelength of laser light to be refracted, the angle of refraction, and the like.

(Liquid Crystal Diffraction Element)

The liquid crystal diffraction element has a liquid crystal layer in which liquid crystal compounds are aligned in a predetermined arrangement, and bends laser light by diffraction.

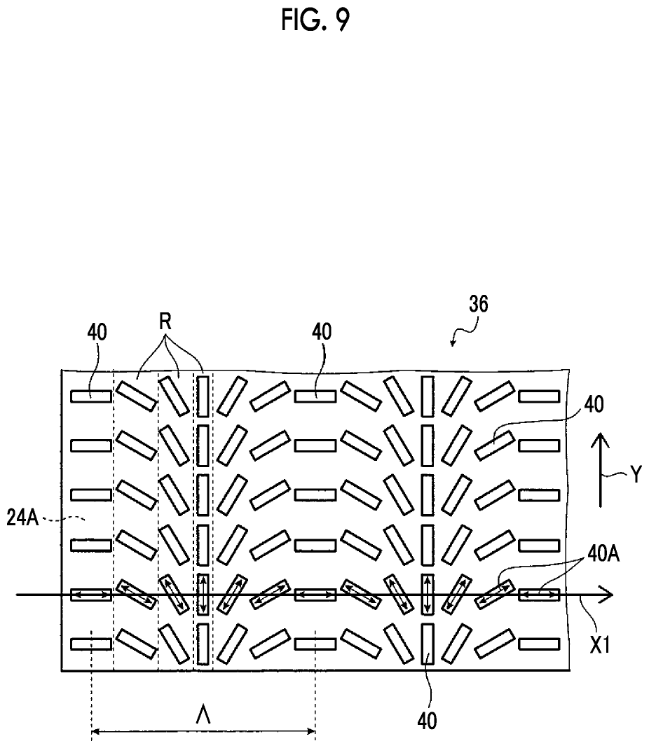

FIG. 8 shows a side view conceptually showing an example of the liquid crystal diffraction element. FIG. 9 is a plan view showing the liquid crystal diffraction element shown in FIG. 8. The plan view refers to a view when the liquid crystal diffraction element is seen from the top, that is, a view when the liquid crystal diffraction element is seen from a thickness direction (a direction in which respective layers (films) are laminated), in FIG. 8. In other words, the plan view is a view when the liquid crystal layer is seen from a direction orthogonal to a main surface.

In addition, in FIG. 9, in order to clearly show the configuration of the liquid crystal diffraction element, only a liquid crystal compound 40 on a surface of an alignment film 32 is shown as the liquid crystal compound 40 in the liquid crystal layer. However, in the thickness direction, as shown in FIG. 8, the liquid crystal layer has a structure in which the liquid crystal compound 40 is stacked on the liquid crystal compound 40 on the surface of the alignment film 32.

A liquid crystal diffraction element 35 shown in FIG. 8 has a support 30, the alignment film 32, and the liquid crystal layer 36. The liquid crystal layer has a predetermined liquid crystal alignment pattern, which is formed of a composition containing the liquid crystal compound, in which an optical axis derived from the liquid crystal compound rotates in one direction in the plane.

[Support]

The support 30 is a film-like material (sheet-like material or plate-like material) that supports the alignment film 32 and the liquid crystal layer 36.

A light transmittance of the support. 30 with respect to light to be diffracted by the liquid crystal diffraction element 35 is preferably 50% or higher, more preferably 70% or higher, and still more preferably 85% or higher.

As a material of the support 30, various resins that are used as a material of a support in the liquid crystal diffraction element may be used.

Specifically, as the material of the support 30, a support having high transparency is preferable, and examples thereof include a polyacrylic resin such as polymethyl methacrylate, a cellulose resin such as cellulose triacetate, a cycloolefin polymer resin, polyethylene terephthalate (PET), polycarbonate, and polyvinyl chloride. The material of the support 30 is not limited to the resin, and glass may be used.

The thickness of the support 30 is not limited, and a thickness with which the alignment film and the liquid crystal layer can be held need only be appropriately set according to the use of the liquid crystal diffraction element 35, the forming material of the support 30, and the like.

The thickness of the support 30 is preferably 1 to 1000 μm, more preferably 3 to 250 μm, and still more preferably 5 to 150 μm.

In the present invention, an aspect in which the support 30 is peeled off and the liquid crystal layer 36 is transferred is also preferably used. That is, a configuration in which the liquid crystal layer 36 is formed on the support 30 and then the support 30 is peeled off and the liquid crystal layer 36 is used as the liquid crystal diffraction element may be employed.

[Alignment Film]

The alignment film 32 is formed on the surface of the support 30.

The alignment film 32 is an alignment film that is used to align the liquid crystal compound 40 in a predetermined liquid crystal alignment pattern in a case where the liquid crystal layer 36 is formed. In the present invention, the liquid crystal layer 36 has a liquid crystal alignment pattern in which an orientation of an optical axis 40A (see FIG. 9) derived from the liquid crystal compound 40 changes while continuously rotating along one direction in the plane (arrow X1 direction, which will be described later).

As the alignment film 32, various well-known films may be used.

Examples of the alignment film include a rubbing-treated film formed of an organic compound such as a polymer, an obliquely deposited film formed of an inorganic compound, a film having a microgroove, and a film formed by lamination of Langmuir-Blodgett (LB) films by a Langmuir-Blodgett's method using an organic compound such as ω-tricosanoic acid, dioctadecylmethylammonium chloride, or methyl stearate.

The rubbing-treated alignment film can be formed by rubbing a surface of a polymer layer with paper or fabric in a certain direction multiple times.

As the material that is used for the alignment film, polyimide, polyvinyl alcohol, a polymer having a polymerizable group disclosed in JP1997-152509A (JP-1109-152509A), a material that is used to form, for example, an alignment film disclosed in JP2005-97377A, JP2005-99228A, and JP2005-128503A are preferable.

In the present invention, as the alignment film, an alignment film in which a material having photo-alignment properties is irradiated with polarized light or non-polarized light, a so-called photo-alignment film is suitably used. That is, in the present invention, a photo-alignment film that is formed by applying a photo-alignment material onto the support 30 is suitably used as the alignment film.

The irradiation with polarized light may be performed in a perpendicular direction or an oblique direction with respect to the photo-alignment film, and the irradiation with non-polarized light may be performed in an oblique direction with respect to the photo-alignment film.

Preferable examples of the photo-alignment material that may be used in the photo-alignment film capable of being used in the present invention include: an azo compound disclosed in JP2006-285197A, JP2007-76839A, JP2007-138138A, JP2007-94071A, JP2007-121721A, JP2007-140465A, JP2007-156439A, JP2007-133184A, JP2009-109831A, JP3883848B, and JP4151746B; an aromatic ester compound disclosed in JP2002-229039A; a maleimide- and/or alkenyl-substituted nadiimide compound having a photo-alignment unit disclosed in JP2002-265541A and JP2002-317013A; a photocrosslinkable silane derivative disclosed in JP4205195B and JP4205198B; a photocrosslinkable polyimide, a photocrosslinkable polyamide, and a photocrosslinkable polyester disclosed in JP2003-520878A, JP2004-529220A, and JP4162850B; and a photodimerizable compound, in particular, a cinnamate compound, a chalcone compound, and a coumarin compound disclosed in JP1997-118717A (JP-H09-118717A), JP1998-506420A (JP-H10-506420A), JP2003-505561 A, WO2010/150748A, JP2013-177561 A, and JP2014-12823A.

Among them, an azo compound, a photocrosslinkable polyimide, a photocrosslinkable polyamide, a photocrosslinkable polyester, a cinnamate compound, and a chalcone compound are suitably used.

The thickness of the alignment film is not limited, and the thickness with which a required alignment function can be obtained need only be appropriately set according to the forming material of the alignment film.

The thickness of the alignment film is preferably 0.01 to 5 ym and more preferably 0.05 to 2 n.

A method of forming the alignment film is not limited, and various well-known methods according to the forming material of the alignment film may be used. An example of the methods include a method in which an alignment film is applied onto the surface of the support 30 and dried, and then the alignment film is exposed to laser light, and an alignment pattern is formed.

FIG. 10 conceptually shows an example of an exposure device that exposes the alignment film to form an alignment pattern.

An exposure device 60 shown in FIG. 10 comprises: a light source 64 including laser 62; a λ/2 plate 65 that changes a polarization direction of laser light M emitted from the laser 62; a polarizing beam splitter 68 that splits the laser light M emitted from the laser 62 into two light rays MA and MB; mirrors 70A and 70B that are disposed on optical paths of the split two light rays MA and MB, respectively; and λ/4 plates 72A and 728.

The light source 64 emits linearly polarized light P₀. The λ/4 plate 72A converts the linearly polarized light P₀ (light ray MA) into right circularly polarized light Px, and the λ/4 plate 72B converts the linearly polarized light P₀ (light ray MB) into left circularly polarized light P_{L}.

The support 30 having the alignment film 32 on which the alignment pattern is not yet formed is disposed at an exposed portion, the two light rays MA and MB intersect and interfere with each other on the alignment film 32, and the alignment film 32 is irradiated with and exposed to the interference light.

Due to the interference in this case, the polarization state of light with which the alignment film 32 is irradiated is periodically changed in interference fringes. As a result, the alignment film having an alignment pattern (hereinafter, also referred to as a patterned alignment film) in which the alignment state is periodically changed can be obtained.

In the exposure device 60, an intersecting angle α between the two light rays MA and MB is changed, so that the period of the alignment pattern can be adjusted. That is, the intersecting angle α is adjusted in the exposure device 60, so that the length of single period over which the optical axis 40A rotates by 180° in the one direction in which the optical axis 40A rotates can be adjusted, in the alignment pattern in which the optical axis 40A derived from the liquid crystal compound 40 continuously rotates along the one direction.

The liquid crystal layer is formed on the alignment film 32 having the alignment pattern in which the alignment state is periodically changed, so that the liquid crystal layer 36 having the liquid crystal alignment pattern in which the optical axis 40A derived from the liquid crystal compound 40 continuously rotates along the one direction can be formed, as will be described later.

In addition, the optical axis of each of the λ/4 plates 72A and 72B rotates by 90°, so that the rotation direction of the optical axis 40A can be reversed.

As described above, the patterned alignment film has a liquid crystal alignment pattern in which the liquid crystal compound is aligned such that the orientation of the optical axis of the liquid crystal compound in the liquid crystal layer formed on the patterned alignment film changes while continuously rotating along at least one direction in the plane. In the patterned alignment film, in a case where an axis along the direction in which the liquid crystal compound is aligned is defined as an alignment axis, it can be said that the patterned alignment film has an alignment pattern in which the orientation of the alignment axis changes while continuously rotating along at least one direction in the plane. The alignment axis of the patterned alignment film can be detected with the measurement of absorption anisotropy. For example, in a case where the patterned alignment film is irradiated with linearly polarized light while rotating the linearly polarized light and the amount of light transmitted through the patterned alignment film is measured, it is observed that a direction in which the amount of light is maximized or minimized is gradually changed along the one direction in the plane.

Note that, in the present invention, the alignment film 32 is provided as a preferable aspect and is not an essential constituent.

For example, the following configuration can also be employed in which the alignment pattern is formed on the support 30 by using methods such as the method of rubbing the support 30 and the method of processing the support 30 with laser light or the like so that the liquid crystal layer 36 has the liquid crystal alignment pattern in which the orientation of the optical axis 40A derived from the liquid crystal compound 40 changes while continuously rotating along at least one direction in the plane. That is, in the present invention, the support 30 may act as the alignment film.

[Liquid Crystal Layer]

The liquid crystal layer 36 is formed on the surface of the alignment film 32.

As described above, in the present invention, the liquid crystal layer is formed of the liquid crystal composition containing the liquid crystal compound.

In a case where the value of the in-plane retardation is set to λ/2, the liquid crystal layer has a function as a general λ/2 plate, that is, a function of giving a phase difference of a half wavelength, that is, 180° to the two linearly polarized light components orthogonal to each other, which are included in the light incident on the liquid crystal layer.

As shown in FIG. 9, the liquid crystal layer has the liquid crystal alignment pattern in which the orientation of the optical axis 40A derived from the liquid crystal compound 40 changes while continuously rotating in the one direction indicated by arrow X1 in the plane of the liquid crystal layer.

The optical axis 40A derived from the liquid crystal compound 40 is an axis having the highest refractive index in the liquid crystal compound 40, that is, a so-called slow axis. For example, in a case where the liquid crystal compound 40 is a rod-like liquid crystal compound, the optical axis 40A is along a rod-shaped major axis direction.

In the following description, “one direction indicated by arrow X1” is also simply referred to as an “arrow X1 direction”. In addition, in the following description, the optical axis 40A derived from the liquid crystal compound 40 is also referred to as “the optical axis 40A of the liquid crystal compound 40” or “the optical axis 40A”.

In the liquid crystal layer, the liquid crystal compounds 40 are two-dimensionally aligned in the plane parallel to the arrow X1 direction and the Y direction orthogonal to the arrow X1 direction, in the liquid crystal layer. In FIG. 8, the Y direction is a direction perpendicular to the paper plane.

FIG. 9 conceptually shows a plan view of the liquid crystal layer 36.

The liquid crystal layer 36 has the liquid crystal alignment pattern in which the orientation of the optical axis 40A derived from the liquid crystal compound 40 changes while continuously rotating along the arrow X1 direction, in the plane of the liquid crystal layer 36.

Specifically, “the orientation of the optical axis 40A of the liquid crystal compound 40 changes while continuously rotating in the arrow X1 direction (the predetermined one direction)” means that an angle formed between the optical axis 40A of each of the liquid crystal compounds 40, which are arranged along the arrow X1 direction, and the arrow X1 direction varies depending on positions in the arrow X1 direction, and the angle formed between the optical axis 40A and the arrow X1 direction sequentially changes from 0° to θ+180° or θ-180° along the arrow X1 direction.

A difference in angle between the optical axes 40A of the liquid crystal compounds 40 adjacent to each other in the arrow X1 direction is preferably 45° or less, more preferably 150 or less, and still more preferably a smaller angle.

Meanwhile, in the liquid crystal compound 40 that forms the liquid crystal layer 36, the liquid crystal compounds 40 of which the optical axes 40A have the same orientation are arranged at an equal interval, in the Y direction orthogonal to the arrow X1 direction, that is, the Y direction orthogonal to the one direction in which the optical axis 40A continuously rotates.

In other words, in the liquid crystal compound 40 that forms the liquid crystal layer 36, the angle formed between the orientation of each optical axis 40A and the arrow X1 direction is the same in all the liquid crystal compounds 40 arranged in the Y direction.

In the present invention, in the liquid crystal alignment pattern of such a liquid crystal compound 40, the length (distance) over which the optical axis 40A of the liquid crystal compound 40 rotates by 180° in the arrow X1 direction in which the orientation of the optical axis 40A changes while continuously rotating in the plane is denoted by a length A of the single period in the liquid crystal alignment pattern. In other words, the length of the single period in the liquid crystal alignment pattern is defined by a distance from 0° to θ+180° of the angle formed between the optical axis 40A of the liquid crystal compound 40 and the arrow X1 direction.

That is, a distance in the arrow X1 direction between the centers of two liquid crystal compounds 40 having the same angle with respect to the arrow X1 direction is denoted by the length A of the single period. Specifically, as shown in FIG. 9, a distance in the arrow X1 direction between the centers of two liquid crystal compounds 40 of which the optical axes 40A have the same orientation as the arrow X1 direction is denoted by the length A of the single period. In the following description, the length A of the single period is also referred to as a “single period A”.

In the present invention, in the liquid crystal alignment pattern of the liquid crystal layer 36, the single period A is repeated in the arrow X1 direction, that is, in the one direction in which the orientation of the optical axis 40A changes while continuously rotating.

As described above, in the liquid crystal layer 36, the angle formed between each optical axis 40A and the arrow X1 direction (the one direction in which the orientation of the optical axis of the liquid crystal compound 40 rotates) is the same in the liquid crystal compounds arranged in the Y direction. A region where the liquid crystal compounds 40 in which the angles formed between the optical axes 40A and the arrow X1 direction are the same are disposed in the Y direction is denoted by a region R.

In this case, it is preferable that the value of an in-plane retardation (Re) of each of the regions R is a half wavelength, that is, λ/2. The in-plane retardation is calculated from the product of a difference Δn in refractive index caused by refractive index anisotropy of the region R and the thickness of the liquid crystal layer 36. Here, the difference in refractive index caused by refractive index anisotropy of the region R in the liquid crystal layer 36 is defined by a difference between a refractive index in a direction of an in-plane slow axis of the region R and a refractive index in a direction orthogonal to the direction of the slow axis. That is, the difference Δn in refractive index caused by refractive index anisotropy of the region R is the same as a difference between a refractive index of the liquid crystal compound 40 in the direction of the optical axis 40A and a refractive index of the liquid crystal compound 40 in a direction perpendicular to the optical axis 40A in a plane of the region R. That is, the difference Δn in refractive index is the same as the difference in refractive index of the liquid crystal compound.

In a case where circularly polarized light is incident on such a liquid crystal layer 36, the light is refracted and the direction of circularly polarized light is converted.

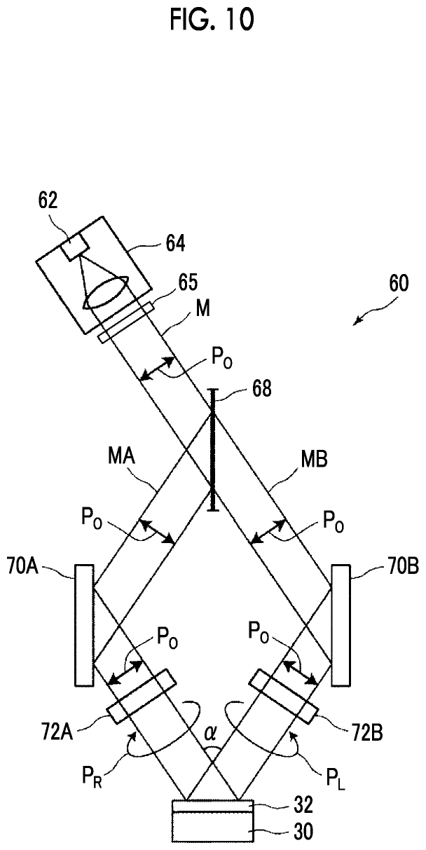

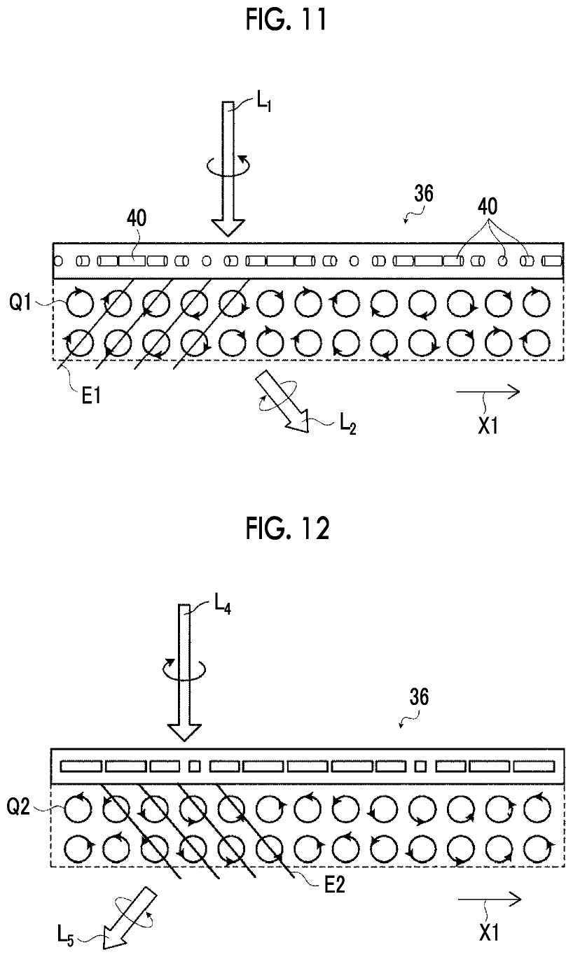

This action is conceptually shown in FIG. 11 by using the liquid crystal layer 36 as an example. In the liquid crystal layer 36, it is assumed that the value of the product of the difference in refractive index of the liquid crystal compound and the thickness of the liquid crystal layer is λ/2.

As shown in FIG. 11, in a case where the value of the product of the difference in refractive index of the liquid crystal compound of the liquid crystal layer 36 and the thickness of the liquid crystal layer is λ/2, when incidence light L₁, which is left circularly polarized light, is incident on the liquid crystal layer 36, the incidence light L₁ passes through the liquid crystal layer 36 to be given a phase difference of 180°, so that transmitted light L₂ is converted into right circularly polarized light.

In addition, when the incidence light L₁ passes through the liquid crystal layer 36, an absolute phase thereof changes according to the orientation of the optical axis 40A of each of the liquid crystal compounds 40. At this time, since the orientation of the optical axis 40A changes while rotating along the arrow X1 direction, the amount of change in the absolute phase of the incidence light L₁ differs depending on the orientation of the optical axis 40A. Further, since the liquid crystal alignment pattern formed on the liquid crystal layer 36 is a pattern that is periodic in the arrow X1 direction, the incidence light L; that has passed through the liquid crystal layer 36 is given an absolute phase Q1 that is periodic in the arrow X1 direction, which corresponds to the orientation of each of the optical axes 40A, as shown in FIG. 11. With this, an equiphase plane E1 that is inclined in a direction opposite to the arrow X1 direction is formed.

Therefore, the transmitted light L₂ is refracted so as to be inclined in a direction perpendicular to the equiphase plane E1 and travels in a direction different from a traveling direction of the incidence light L₁. In this way, the incidence light L₁ of the left circularly polarized light is converted into the transmitted light L₂ of right circularly polarized light that is inclined by a certain angle in the arrow X1 direction with respect to an incidence direction.

On the other hand, as conceptually shown in FIG. 12, in a case where the value of the product of the difference in refractive index of the liquid crystal compound of the liquid crystal layer 36 and the thickness of the liquid crystal layer 36 is λ/2, when incidence light L₄ of right circularly polarized light is incident on the liquid crystal layer 36, the incidence light L₄ passes through the liquid crystal layer 36 to be given a phase difference of 180° and is converted into transmitted light L₅ of left circularly polarized light.

In addition, when the incidence light L₄ passes through the liquid crystal layer 36, an absolute phase thereof changes according to the orientation of the optical axis 40A of each of the liquid crystal compounds 40. At this time, since the orientation of the optical axis 40A changes while rotating along the arrow X1 direction, the amount of change in the absolute phase of the incidence light L₄ differs depending on the orientation of the optical axis 40A. Further, since the liquid crystal alignment pattern formed on the liquid crystal layer 36 is a pattern that is periodic in the arrow X1 direction, the incidence light L₄ that has passed through the liquid crystal layer 36 is given an absolute phase Q2 that is periodic in the arrow X1 direction, which corresponds to the orientation of each of the optical axes 40A, as shown in FIG. 12.

Here, since the incidence light L₄ is right circularly polarized light, the absolute phase Q2 that is periodic in the arrow X1 direction, which corresponds to the orientation of the optical axis 40A, is opposite to the incidence light L₁ of left circularly polarized light. As a result, in the incidence light L₄, an equiphase plane E2 that is inclined in the arrow X1 direction opposite to that of the incidence light L₁ is formed.

Therefore, the incidence light L₄ is refracted so as to be inclined in a direction perpendicular to the equiphase plane E2 and travels in a direction different from a traveling direction of the incidence light L₄. In this way, the incidence light L₄ is converted into the transmitted light L₅ of left circularly polarized light that is inclined by a certain angle in a direction opposite to the arrow X1 direction with respect to an incidence direction.

In the liquid crystal layer 36, the value of the in-plane retardation of each of a plurality of regions R is preferably a half wavelength of the incidence light.

Here, the single period A of the liquid crystal alignment pattern formed on the liquid crystal layer 36 is changed, so that refraction angles of the transmitted light L₂ and L₅ can be adjusted. Specifically, since light rays that have passed through the liquid crystal compounds 40 adjacent to each other more strongly interfere with each other as the single period A of the liquid crystal alignment pattern decreases, the transmitted light L₂ and L₅ can be greatly refracted.

In addition, refraction angles of the transmitted light L₂ and L₅ with respect to the incidence light L₁ and L₄ vary depending on the wavelengths of the incidence light L₁ and L₄ (transmitted light L₂ and L₅). Therefore, the single period A of the liquid crystal alignment pattern need only be set according to the wavelength of laser light emitted by the laser light source and the angle at which the laser light is incident on the user U.

Further, the rotation direction of the optical axis 40A of the liquid crystal compound 40, which rotates along the arrow X1 direction, is made reversed, the refraction direction of the transmitted light can be reversed.

The liquid crystal layer 36 consists of a cured layer of a liquid crystal composition containing a rod-like liquid crystal compound or a disk-like liquid crystal compound, and has a liquid crystal alignment pattern in which an optical axis of the rod-like liquid crystal compound or an optical axis of the disk-like liquid crystal compound is aligned as described above.

The alignment film 32 is formed on the support 30 and the liquid crystal composition is applied and cured on the alignment film 32, so that the liquid crystal layer 36 consisting of the cured layer of the liquid crystal composition can be obtained. Note that, although the liquid crystal layer 36 functions as a so-called λ/2 plate, the present invention also includes an aspect in which a laminate integrally comprising the support 30 and the alignment film 32 functions as the λ/2 plate.

In addition, the liquid crystal composition that is used to form the liquid crystal layer 36 contains a rod-like liquid crystal compound or a disk-like liquid crystal compound, and may further contain other components such as a leveling agent, an alignment control agent, a polymerization initiator, a crosslinking agent, and an alignment assistant. Further, the liquid crystal composition may contain a solvent.

Further, the liquid crystal layer 36 can be made in various forms of layer substantially having a function of a λ/2 plate, that is, a function of converting right circularly polarized light into left circularly polarized light and converting left circularly polarized light into right circularly polarized light.

—Rod-Like Liquid Crystal Compound—

As the rod-like liquid crystal compound, azomethine compounds, azoxy compounds, cyanobiphenyl compounds, cyanophenyl ester compounds, benzoate compounds, phenyl cyclohexanecarboxylate compounds, cyanophenylcyclohexane compounds, cyano-substituted phenylpyrimidine compounds, alkoxy-substituted phenylpyrimidine compounds, phenyldioxane compounds, tolan compounds, and alkenylcyclohexylbenzonitrile compounds are preferably used. As the rod-like liquid crystal compound, not only the low-molecular-weight liquid crystal molecules as described above but also high-molecular-weight liquid crystal molecules may be used.

It is preferable that the alignment of the rod-like liquid crystal compound is immobilized by polymerization, and as the polymerizable rod-like liquid crystal compound, the compounds disclosed in Makromol. Chem., Vol. 190, p. 2255 (1989), Advanced Materials, Vol. 5, p. 107 (1993), U.S. Pat. Nos. 4,683,327A, 5,622,648A, 5,770,107A, WO95/22586A, WO95/24455A, WO97/00600A, WO98/23580A, WO98/52905A, JP1989-272551A (JP-H01-272551A), JP1994-16616A (JP-H06-16616A), JP1995-110469A (JP-107-110469A), JP1999-80081A (JP-H11-80081A), JP2001-64627A, and the like may be used. Further, as the rod-like liquid crystal compound, for example, compounds disclosed in JP1999-513019A (JP-H11-513019A) and JP2007-279688A may be preferably used.

—Disk-Like Liquid Crystal Compound—

As the disk-like liquid crystal compound, for example, compounds disclosed in JP2007-108732A and JP2010-244038A may be preferably used.

In a case where the disk-like liquid crystal compound is used for the liquid crystal layer, the liquid crystal compound 40 stands in the thickness direction in the liquid crystal layer, and the optical axis 40A derived from the liquid crystal compound is defined as an axis perpendicular to a disk plane, a so-called fast axis.

In addition, the liquid crystal diffraction element may have a configuration in which a plurality of liquid crystal layers are provided. With the plurality of liquid crystal layers provided, the diffraction efficiency can be improved. In a case where the plurality of liquid crystal layers are provided, the single periods A of the liquid crystal alignment patterns of the liquid crystal layers may be the same or different from each other. Alternatively, the liquid crystal alignment pattern may be different for each liquid crystal layer.

Here, the length of the single period A in the alignment pattern of the liquid crystal layer is not particularly limited, and the single period A of the liquid crystal alignment pattern need only be set according to the wavelength of laser light emitted by the laser light source and the angle at which the laser light is incident on the user U.

In consideration of the accuracy of the liquid crystal alignment pattern and the like, the single period A in the liquid crystal alignment pattern of the liquid crystal layer is preferably 0.1 μm or more.

<<Method of Forming Liquid Crystal Layer>>

A method of forming the liquid crystal layer includes, for example, a step of applying a liquid crystal composition containing the prepared liquid crystal compound onto the alignment film and a step of curing the applied liquid crystal composition.

The liquid crystal composition need only be prepared by using a conventionally well-known method. In addition, for the application of the liquid crystal composition, various well-known methods that are used to apply liquid, such as bar coating, gravure coating, and spray coating, may be used. Further, for the coating thickness of the liquid crystal composition (the thickness of the coating film), the coating thickness with which a liquid crystal layer having a desired thickness can be obtained need only be appropriately set according to composition of the liquid crystal composition and the like.

Here, since the alignment pattern is formed on the alignment film, the liquid crystal compound of the liquid crystal composition applied onto the alignment film is aligned along with the alignment pattern (anisotropically periodic pattern) of the alignment film.

The liquid crystal composition is optionally dried and/or heated and then cured. The liquid crystal composition need only be cured by using a well-known method such as photopolymerization or thermal polymerization. For the polymerization, photopolymerization is preferable. For the light irradiation, ultraviolet rays are preferably used. The irradiation energy is preferably 20 mJ/cm² to 50 J/cm² and more preferably 50 to 1500 mJ/cm². In order to promote a photopolymerization reaction, the light irradiation may be performed under heating conditions or the nitrogen atmosphere. The wavelength of ultraviolet rays with which the liquid crystal composition is irradiated is preferably 250 to 430 nm.

The liquid crystal composition is cured, so that the liquid crystal compound in the liquid crystal composition is immobilized in a state in which the liquid crystal compound is aligned along with the alignment pattern of the alignment film (liquid crystal alignment pattern). As a result, a liquid crystal layer having a liquid crystal alignment pattern in which an orientation of an optical axis derived from the liquid crystal compound changes while continuously rotating along at least one direction in the plane is formed. The liquid crystal alignment pattern of the liquid crystal layer will be described in detail later.

Note that, at the time when the liquid crystal layer is completed, the liquid crystal compound may not exhibit liquid crystal properties. For example, the polymerizable liquid crystal compound may lose the liquid crystal properties with the increase in molecular weight thereof caused by a curing reaction.

Alternatively, the liquid crystal layer may be formed by multi-layer coating of the liquid crystal composition on the alignment film. The multi-layer coating refers to repetition of, first, applying a liquid crystal composition for a first layer onto the alignment film, heating and cooling the applied liquid crystal composition, and then curing the liquid crystal composition with ultraviolet rays to prepare the liquid crystal immobilized layer, and then of applying a liquid crystal composition for second and subsequent layers to the liquid crystal immobilized layer by overlapping coating, and heating and cooling the applied liquid crystal composition in the same manner, and then curing the liquid crystal composition with ultraviolet rays. With the liquid crystal layer formed by the multi-layer coating, the total thickness of the liquid crystal layer can be increased. In addition, the alignment direction of the alignment film is reflected from a lower surface to an upper surface of the liquid crystal layer, even with the liquid crystal layer having the total thickness increased.

) Alternatively, the liquid crystal diffraction element may have a layer other than the support, the alignment film, and the liquid crystal layer.

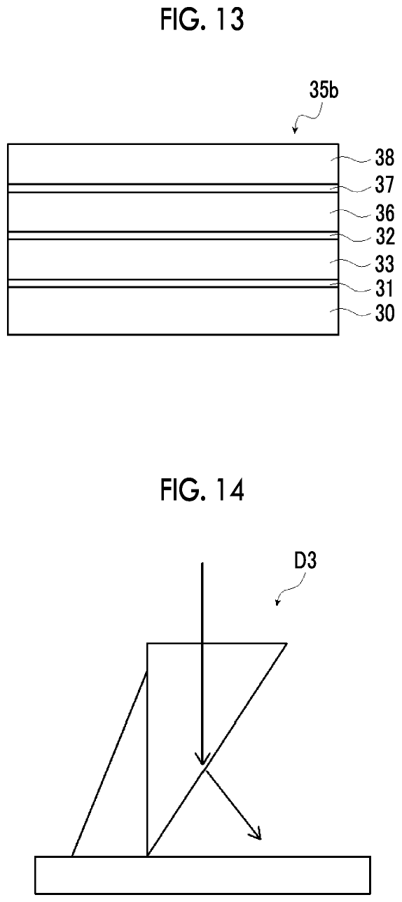

For example, as in a liquid crystal diffraction element 35b shown in FIG. 13, the support 30, an alignment film 31, a first λ/4 plate 33, the alignment film 32, the liquid crystal layer 36, an alignment film 37, and a second λ/4 plate 38 may be provided in this order. The alignment film 31 is an alignment film for forming the first λ/4 plate 33, and the alignment film 37 is an alignment film for forming the second λ/4 plate 38.

For example, in a case where laser light emitted by the laser light source 14 is linearly polarized light, when the laser light is incident on the liquid crystal diffraction element 35b from the second λ/4 plate 38 side, the second λ/4 plate 38 converts the linearly polarized light into circularly polarized light, the liquid crystal layer 36 reverses the revolution direction of the circularly polarized light and diffracts the laser light, and the first λ/4 plate 33 converts the circularly polarized light transmitted through the liquid crystal layer 36 into linearly polarized light. Therefore, linearly polarized light is incident on the user U from a direction inclined with respect to the surface of the user U. With the light incident on the user U made P-polarized, reflection on the skin surface can be restrained.

As the first λ/4 plate 33 and the second λ/4 plate 38, a well-known λ/4 plate need only be used.

Further, in the example shown in FIG. 13, although a configuration is employed in which the alignment film 31 is provided between the support 30 and the first λ/4 plate 33 and the alignment film 37 is provided between the liquid crystal layer 36 and the second λ/4 plate 38, a configuration in which the alignment film 31 and/or the alignment film 37 is not provided, a configuration in which the support 30 and the first λ/4 plate 33 are bonded to each other with an adhesive layer, and/or a configuration in which the liquid crystal layer 36 and the second λ/4 plate 38 are bonded to each other with an adhesive layer may also be employed.

Here, the liquid crystal diffraction element is disposed so as to bend the incident laser light source to the light receiving unit side along the flow direction of the blood vessel. As shown in FIGS. 11 and 12, the refraction direction of light by the liquid crystal diffraction element is one direction in the plane (arrow X1 direction) in which the orientation of the optical axis 40A derived from the liquid crystal compound 40 changes while continuously rotating. Therefore, the liquid crystal diffraction element is disposed such that the arrow X1 direction is along the flow direction of the blood vessel.

(Prism)

A prism may also be used as the light bending member 18.

The prism is a polyhedron made of a transparent medium, such as glass and crystal, having a predetermined refractive index, and refracts incidence light, thereby bending laser light. As the prism that is used as the light bending member 18, a conventionally well-known prism may be appropriately used as long as laser light can be bent to a desired angle. As an example, a prism D3 shown in FIG. 14 has a transparent polyhedron having a right angled triangular cross section.

(Mirror)

A mirror may also be used as the light bending member 18.

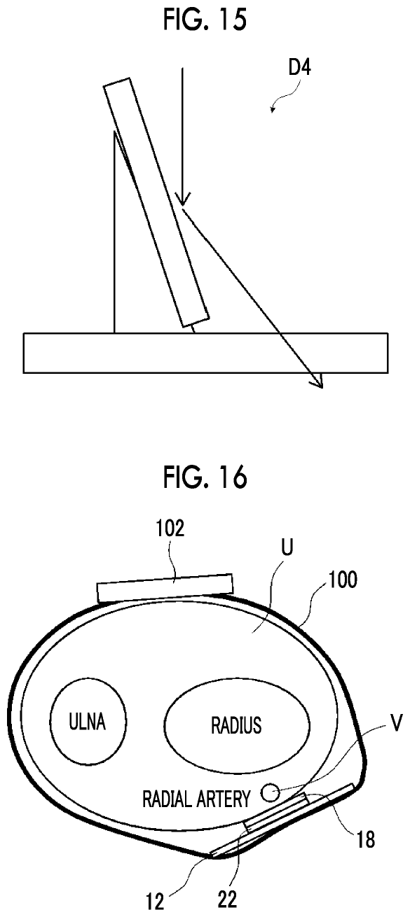

In the example shown in FIG. 15, a mirror D4 is disposed at a predetermined angle, and the light incident on the mirror is specularly reflected and travels in a direction different from the incidence direction. With this, the laser light is bent.

The angle of the mirror is adjusted, so that the laser light can be bent to a desired angle.

As the light bending member 18, a prism sheet, a lens sheet, and a liquid crystal diffraction element are preferable from the viewpoint that it is not easily damaged even in a case of being strongly fixed when the flow rate measuring device is worn by the user, the followability with respect to body movement is high, and the like, and a liquid crystal diffraction element is more preferable from the viewpoint of diffraction efficiency and the like.

<Light Condensing Member>

The light condensing member 20 is used to bend (condense) the laser light reflected in the body of the user U toward the light receiving unit.

As the light condensing member 20, various members, a convex lens, a Fresnel lens sheet, and the like that are used as the above-described light bending member 18 may be used.

Here, the flow rate measuring device of the embodiment of the present invention preferably has a holding mechanism that keeps a distance between the light bending member and the object constant.

An example of the holding mechanism included in the flow rate measuring device of the embodiment of the present invention will be described with reference to FIGS. 16 to 18.

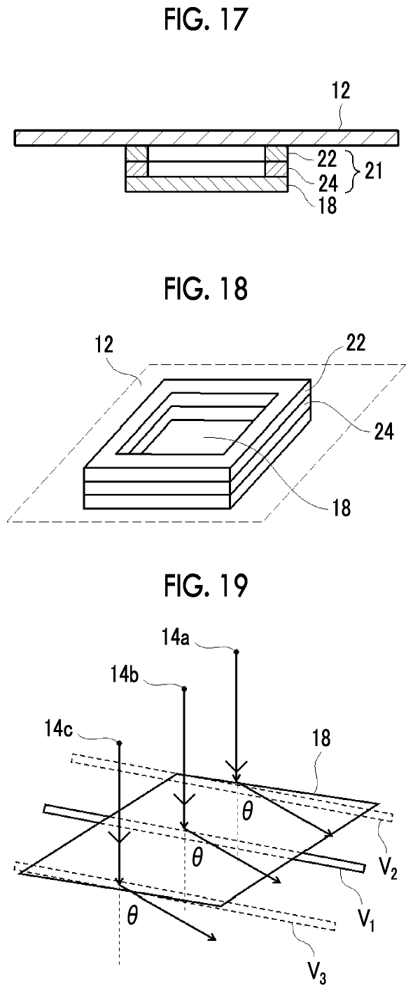

FIG. 16 is a cross-sectional view schematically showing another example of the flow rate measuring device of the embodiment of the present invention. FIG. 17 is a side view showing a part of the flow rate measuring device shown in FIG. 16. FIG. 18 is a perspective view of FIG. 17.

The flow rate measuring device shown in FIG. 16 includes the substrate 12, a frame 24, the light bending member 18, a band 100, and a display 102. In FIGS. 16 to 18, the laser light source, the light receiving unit 16, and the like are not shown.

Since the substrate 12 and the light bending member 18 have the same configurations as the substrate 12 and the light bending member 18 of the flow rate measuring device 10 shown in FIG. 1, the description thereof will be omitted.

In the example shown in FIG. 16, a band-like band 100 is wrapped around the wrist of the user U, and the substrate 12, the frame 24, and the light bending member 18 are held near the radial artery V between the band 100 and the user U.

The display 102 is installed on the band 100. The display 102 displays information on the measured blood flow velocity or information on the blood pressure (relative value of blood pressure) calculated from the blood flow velocity, the stress level of the person to be measured, and the like.

As shown in FIGS. 17 and 18, an elastic member 22 and the frame 24 each of which has an outer shape having substantially the same size as that of the outer periphery of the light bending member 18 are provided between the substrate 12 and the light bending member 18.

The frame 24 has a rectangular parallelepiped outer shape and has a quadrangular opening portion penetrating in a direction perpendicular to the surface of the substrate 12. The shape of the opening portion and an opening surface of the frame 24 is not limited to a quadrangular shape, and may be a circular shape or a polygonal shape.

The light bending member 18 is fixed to one opening surface of the frame 24. The elastic member 22 is disposed on the other opening surface.

The frame 24 is not particularly limited, and a frame made of resin, metal, or the like may be used.

The elastic member 22 has a rectangular parallelepiped outer shape and has a quadrangular opening portion penetrating in the direction perpendicular to the surface of the substrate 12. The shape of the opening portion and an opening surface of the elastic member 22 is not limited to a quadrangular shape, and may be a circular shape or a polygonal shape.

The elastic member 22 need only have elasticity, and a porous material such as urethane sponge, a spring, rubber, a pressure-sensitive adhesive layer having elasticity (pressure-sensitive adhesive gel sheet), or the like may be used.

Although not shown, the laser light source and the light receiving unit are disposed on the substrate 12 in the opening portion of the elastic member 22 (frame 24).

In such a configuration, the band 100, the frame 24, and the elastic member 22 correspond to the holding mechanism that keeps the distance between the light bending member 18 and the user U constant.

That is, the light bending member 18 bonded to the frame 24 is held at a predetermined position of the user U integrally with the substrate 12 and the like by the band 100. Further, since the light bending member 18 is biased toward the user U by the elastic member 22, the light bending member 18 follows the surface of the user U so as to be in contact with the surface of the user U even in a case where the distance between the substrate 12 and the user U changes due to body movement.

There is a concern that in a case where the laser light source itself is tilted as described above although the distance between the user U and the laser light source is likely to deviate in the case of the wearable measuring device, the blood flow cannot be measured properly because the point at which laser light is incident on the user changes and the distance from the incidence point to the emission point is likely to change when the distance between the user U and the laser light source deviates.

In response to this, a configuration is employed in which the light bending member 18 that bends laser light is provided and the holding mechanism keeps the distance between the light bending member 18 and the user U constant, so that it is possible to restrain the distance from the incidence point to the emission point from changing and to measure the blood flow properly even in a case where the distance between the user U and the laser light source deviates, while a configuration is employed in which the laser light is incident on the surface of the user U from the oblique direction.

Further, there is a concern that the blood vessel may be compressed in a case where the flow rate measuring device is strongly fixed to the wrist of the user U and the blood flow may not be measured normally. Therefore, there is a concern that the blood vessel may be compressed and the blood flow may not be measured normally, for example, in a case where the band is tightly wrapped around the wrist of the user U. In response to this, a configuration is employed in which the light bending member 18 is biased toward the user U by using the elastic member 22, so that it is possible to restrain the position of the light bending member 18 from deviating due to body movement even in a case where the band is not tightly wrapped around the wrist of the user U.

In the example shown in FIG. 17, the light bending member 18 and the elastic member 22 are laminated via the frame 24, but the present invention is not limited thereto, and the light bending member 18 may be laminated directly to the elastic member 22.

Further, in the flow rate measuring device of the embodiment of the present invention, it is also preferable that the light source unit includes a substrate and a plurality of laser oscillating elements provided on the substrate.

FIG. 19 is a perspective view schematically showing another example of the flow rate measuring device of the embodiment of the present invention. In FIG. 19, the substrate, the light receiving element, and the like are not shown.

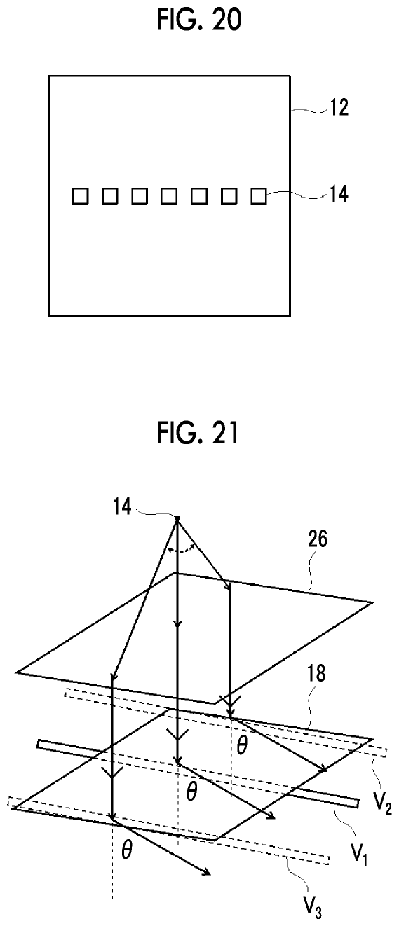

FIG. 20 shows the configuration of the light source unit in the example shown in FIG. 19.

As shown in FIG. 20, the light source unit includes the substrate 12 and a plurality of the laser light sources 14 provided on the substrate 12. In the example shown in FIG. 20, seven laser light sources 14 are arranged in one direction.

As shown in FIG. 19, the light source unit is disposed such that the arrangement direction of the plurality of laser light sources is orthogonal to the flow direction of the blood vessel V to be measured.

The laser light emitted from the plurality of laser light sources is incident on the light bending member 18, and is bent at an angle θ in the flow direction of the blood vessel V and emitted into the body of the user U.

Here, with the configuration in which the light source unit has the plurality of laser light sources, it is possible to irradiate the blood vessel V with laser light even in a case where the relative position between the flow rate measuring device and the wrist of the user U deviates due to body movement.

Specifically, for example, as shown in FIG. 19, in a case where the blood vessel V is at a blood vessel position V₁, laser light that is emitted by a laser light source 14b located at a position corresponding to the blood vessel position V₁ out of the plurality of laser light sources 14 is emitted to the blood vessel V. In a case where the blood vessel position moves relatively from V₁ to V₂ due to body movement, laser light that is emitted by a laser light source 14a located at a position corresponding to the blood vessel position V₂ out of the plurality of laser light sources 14 is emitted to the blood vessel V. Further, in a case where the blood vessel position moves relatively to the blood vessel position V₃ due to body movement, laser light that is emitted by a laser light source 14c located at a position corresponding to the blood vessel position V₃ out of the plurality of laser light sources 14 is emitted to the blood vessel V.

Note that, in a case where the light source unit has the plurality of laser light sources, all the laser light sources 14 may emit laser light, or only the laser light source 14 located at a position corresponding to the blood vessel position may emit laser light after searching for the blood vessel position. Alternatively, in order to search for the blood vessel position, all the laser light sources 14 may emit laser light once, and then only the laser light source 14 located at the position corresponding to the blood vessel position may emit laser light.

Further, the number of laser light sources included in the light source unit is not limited to seven, and may be two to six or eight or more.

Further, in the flow rate measuring device of the embodiment of the present invention, the light source unit may have a laser oscillating element, an alignment mechanism that performs scanning with laser light emitted from the laser oscillating element, and an optical member that makes an incidence angle of the laser light with which the scanning is performed by the alignment mechanism, on the light bending member constant.

FIG. 21 shows an example of a flow rate measuring device having such a configuration.

The flow rate measuring device shown in FIG. 21 includes the laser light source 14, an optical member 26, and the light bending member 18. In FIG. 21, the substrate, the light receiving element, and the like are not shown. Further, in FIG. 21, although the alignment mechanism is not shown, it is shown that the scanning is performed with laser light emitted from the laser light source 14 by the alignment mechanism.

As shown in FIG. 21, the alignment mechanism performs scanning with the laser light emitted from the laser light source 14 in a direction orthogonal to the flow direction of the blood vessel V. The light with which scanning is performed is incident on the optical member 26.

The alignment mechanism is not particularly limited, and an alignment mechanism that is used to perform scanning with light such as a mechanism that performs scanning with light by rotating a polygon mirror or a micro electro mechanical systems (MEMS) mirror may be appropriately used.

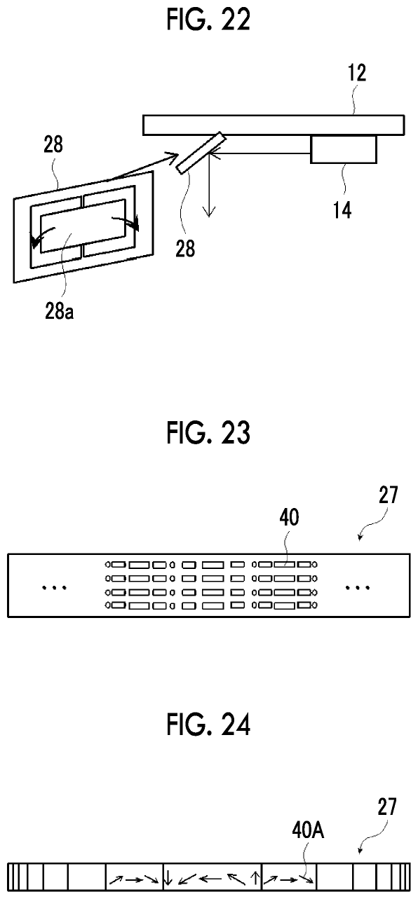

As an example, FIG. 22 is an example of a light source unit using a MEMS mirror as an alignment mechanism 28. The light source unit shown in FIG. 22 has the substrate 12, a laser light source 14 disposed on the substrate 12, and the alignment mechanism (MEMS mirror) 28 disposed on the substrate 12.

The laser light source 14 is an end surface emitting laser that irradiates a mirror 28a of the MEMS mirror 28 with light in a direction parallel to the substrate 12.

The MEMS mirror 28 drives the mirror 28a to swing by electromagnetic drive. In a case where the light emitted from the laser light source 14 is reflected by the mirror 28a, the direction of the laser light emitted by the MEMS mirror 28 varies depending on the angle of the mirror 28a to be driven. That is, the MEMS mirror 28 changes the direction of the laser light so as to scan the optical member 26 with the laser light.

As described above, the scanning direction by the alignment mechanism 28 is a direction orthogonal to the flow direction of the blood vessel V.

The optical member 26 is used to bend the traveling direction of the laser light incident for the scanning by the alignment mechanism 28 so that the laser light is incident on the light bending member 18 from a predetermined direction. In the example shown in FIG. 21, the optical member 26 bends the traveling direction of the laser light so that the laser light is incident on the light bending member 18 from a direction perpendicular to the surface of the light bending member 18.

Here, as shown in FIG. 21, it can be said that the laser light with which scanning is performed by the alignment mechanism 28 is diffused light of which the traveling direction spreads in a fan shape. On the other hand, it can be said that in a case where the optical member 26 bends the laser light so that the laser light is incident on the light bending member 18 from a predetermined direction, the optical member 26 makes the laser light parallel light.

In other words, since the angle at which the laser light is incident on the optical member 26 differs depending on the position in the scanning direction, the optical member 26 bends the laser light at different angles according to the position in the scanning direction. Specifically, in the scanning direction, the angle at which the laser light is bent is increased as the distance from the position of the laser light source (the position where the alignment mechanism 28 is irradiated with the laser light) is increased.

Examples of such an optical member 26 include a gradient index lens in which the refractive index is distributed from the center to the outer peripheral portion of the lens, and a liquid crystal lens, which is a liquid crystal diffraction element using a liquid crystal compound, in which the diffraction angle is distributed.

The liquid crystal lens will be described in detail later.

All the laser light bent by the optical member 26 and incident on the light bending member 18 is bent by the light bending member 18 at an angle θ in the flow direction of the blood vessel V and emitted into the body of the user U.

Here, with the configuration in which scanning is performed by the alignment mechanism with the laser light, the blood vessel V can be irradiated with the laser light even in a case where the relative position between the flow rate measuring device and the wrist of the user U deviates due to body movement.

Specifically, for example, as shown in FIG. 21, in any cases such as a case where the blood vessel V is at the blood vessel position V₁, a case where the blood vessel position moves relatively from V₁ to V₁ due to body movement, and a case where the blood vessel position moves relatively to V₃ due to body movement, the blood vessel V is irradiated with laser light because the laser light emitted from the laser light source 14 spreads in a plane shape.

(Liquid Crystal Lens)

Hereinafter, the liquid crystal lens will be described with reference to FIGS. 23 and 24.

The liquid crystal lens has a liquid crystal layer in which liquid crystal compounds are aligned in a predetermined arrangement, bends the laser light by diffraction, and has a diffraction angle varying depending on the position.

FIG. 23 shows a side view conceptually showing an example of a liquid crystal layer 27 included in the liquid crystal lens. FIG. 24 shows a plan view of FIG. 23. In FIG. 24, in order to clearly show the configuration of the liquid crystal layer 27, only the liquid crystal compound 40 on the surface of the alignment film is shown as the liquid crystal compound 40 in the liquid crystal layer 27. However, the liquid crystal layer 27 has a structure in which the liquid crystal compounds 40 are stacked as shown in FIG. 23 in the thickness direction.

The liquid crystal layer 27 included in the liquid crystal lens has a predetermined liquid crystal alignment pattern, which is formed of a composition containing the liquid crystal compound 40, in which the optical axis (optical axis 40A) derived from the liquid crystal compound rotates in one direction in the plane. In a case where the length over which the orientation of the optical axis 40A rotates by 180° along the one direction in the plane is defined as a single period, the liquid crystal layer 27 has a region having a length different from the length of the single period of the liquid crystal alignment pattern, in the plane of the liquid crystal layer 27.

In the examples shown in FIGS. 23 and 24, the length of the single period of the liquid crystal alignment pattern becomes shorter from the center toward the peripheral portions on both sides. As described above, the shorter the length of the single period of the liquid crystal alignment pattern, the greater the diffraction of light (the larger the diffraction angle). Therefore, in the liquid crystal layer 27 shown in FIGS. 23 and 24, the diffraction angle at the central portion is small, and the diffraction angle is increased toward the peripheral portion.

The liquid crystal layer 27 included in the liquid crystal lens has basically the same configuration as that of the liquid crystal layer 36 of the liquid crystal diffraction element 35 described above, except that the liquid crystal layer 27 has a region having a length different from the length of the single period of the liquid crystal alignment pattern, in the plane of the liquid crystal layer. Therefore, the description except for the point will be omitted.

With the configuration in which the length of the single period of the liquid crystal alignment pattern becomes shorter from the center toward the peripheral portions on both sides, the liquid crystal layer 27 included in the liquid crystal lens can bend the laser light greatly as the distance from the position of the laser light source (the position where the alignment mechanism 28 is irradiated with the laser light) is increased in the scanning direction of the laser light.

The liquid crystal layer 27 included in the liquid crystal lens is formed by forming the liquid crystal compound 40 on an alignment film having an alignment pattern that is used to align the liquid crystal compound 40 in the above-described liquid crystal alignment pattern.

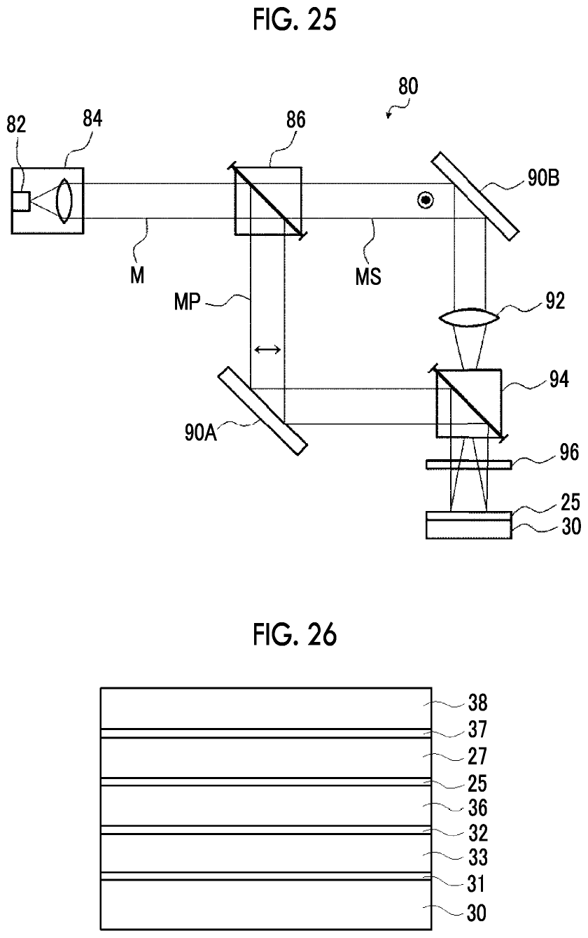

FIG. 25 conceptually shows an example of an exposure device that forms such an alignment pattern on the alignment film.

An exposure device 80 includes: a light source 84 provided with laser 82; a polarizing beam splitter 86 that splits laser light M from the laser 82 into S-polarized light MS and P-polarized light MP; a mirror 90A disposed on an optical path of the P-polarized light MP and a mirror 90B disposed on an optical path of the S-polarized light MS; a lens 92 disposed on the optical path of the S-polarized light MS; a polarizing beam splitter 94; and a λ/4 plate 96.

The P-polarized light MP that is split by the polarizing beam splitter 86 is reflected by the mirror 90A to be incident on the polarizing beam splitter 94. On the other hand, the S-polarized light MS that is split by the polarizing beam splitter 86 is reflected by the mirror 90B and is condensed by the lens 92 to be incident on the polarizing beam splitter 94.

The P-polarized light MP and the S-polarized light MS are multiplexed by the polarizing beam splitter 94, are converted into right circularly polarized light and left circularly polarized light by the λ/4 plate 96 according to the polarization direction, and are incident on the alignment film 25 on the support 30.

Here, the polarization state of light with which the alignment film 25 is irradiated is periodically changed in interference fringes due to interference between the right circularly polarized light and the left circularly polarized light. The intersecting angle between the right circularly polarized light and the left circularly polarized light changes from the inside toward the outside. Therefore, an exposure pattern in which the pitch changes from the inside toward the outside can be obtained. As a result, in the alignment film 25, an alignment pattern in which the length of the single period of the alignment pattern changes can be obtained.

In the exposure device 80, the length of the single period of the liquid crystal alignment pattern in which the optical axis of the liquid crystal compound 40 continuously rotates by 180° can be controlled by changing the optical power of the lens 92 (the F number of the lens 92), the focal length of the lens 92, the distance between the lens 92 and the alignment film 32, and the like.

In addition, the optical power of the lens 92 (the F number of the lens 92) is adjusted, so that the length of the single period of the liquid crystal alignment pattern in one direction in which the optical axis continuously rotates can be changed. Specifically, the length of the single period of the liquid crystal alignment pattern in one direction in which the optical axis continuously rotates can be changed by using the spread angle of light spread by the lens 92, which interferes with parallel light. More specifically, since the light is approximated to parallel light in a case where the optical power of the lens 92 is weak, the length of the single period of the liquid crystal alignment pattern gradually decreases from the inside toward the outside, and the F number increases. Conversely, in a case where the optical power of the lens 92 is stronger, the length A of the single period of the liquid crystal alignment pattern rapidly decreases from the inside toward the outside, and the F number decreases.

Here, the liquid crystal layer 27 of the liquid crystal lens, which is the optical member 26, may be laminated with the liquid crystal layer 36 of the liquid crystal diffraction element, which is the light bending member 18.

A laminate shown in FIG. 26 has the support 30, the alignment film 31, the first λ/4 plate 33, the alignment film 32, the liquid crystal layer 36, the alignment film 25, the second liquid crystal layer 27, the alignment film 37, and the second λ/4 plate 38 in this order. The second liquid crystal layer 27 is a liquid crystal layer of the liquid crystal lens which is the optical member 26. Further, the alignment film 25 is an alignment film for forming the second liquid crystal layer 27. The other layers have basically the same configuration as that of the laminate shown in FIG. 13.

Although the flow rate measuring device of the embodiment of the present invention has been described above in detail, the present invention is not limited to the above-described example, and various improvements and changes may be made without departing from the gist of the present invention.

### EXAMPLES

Hereinafter, the characteristics of the present invention will be described in detail using examples. Materials, chemicals, used amounts, material amounts, ratios, treatment details, treatment procedures, and the like shown in the following examples can be appropriately changed without departing from the gist of the present invention. Therefore, the scope of the present invention should not be construed as being limited to specific examples shown below.

### Example 1

[Preparation of Liquid Crystal Diffraction Element]

(Formation of Alignment Film 1)

As the support, a triacetyl cellulose (TAC) film (ZRG40 manufactured by Fujifilm Corporation, a phase difference of 0) was prepared.

The following coating liquid for forming alignment film 1 was applied onto the support by spin coating. The support on which the coating film of the coating liquid for forming alignment film 1 was formed was dried using a hot plate at 60° C. for 60 seconds, so that an alignment film 1 was formed.

Coating Liquid for Forming Alignment Film 1

| | |
|---|---|
| The following material A for photo-alignment | 1.00 part by mass |
| Water | 16.00 parts by mass |
| Butoxyethanol | 42.00 parts by mass |
| Propylene glycol monomethyl ether | 42.00 parts by mass |

Material A for Photo-Alignment-

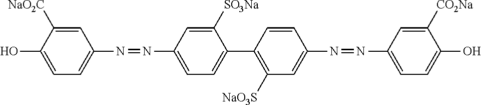

(Exposure of Alignment Film 1)

The alignment film 1 was irradiated with polarized ultraviolet rays (100 mJ/cm, using an ultra-high pressure mercury lamp), so that the alignment film 1 was exposed.

(Formation of First λ/4 Plate)

The following coating liquid for forming λ/4 layer 1 was prepared as a liquid crystal composition for forming the first λ/4 plate (hereinafter, referred to as a λ/4 layer 1).

Coating Liquid for Forming λ/4 layer 1

| | |
|---|---|
| Liquid crystal compound L-1 | 100.00 parts by mass |
| Polymerization initiator (Irgacure (registered trademark) 907, manufactured by BASF SE) | 3.00 parts by mass |
| Photosensitizer (KAYACURE DETX-S, manufactured by Nippon Kayaku Co., Ltd.) | 1.00 part by mass |
| Leveling agent T-1 | 0.08 parts by mass |
| Methyl ethyl ketone | 193.00 parts by mass |

Liquid Crystal Compound L-1- 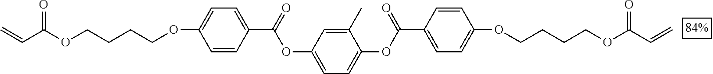 [SMILES: C=CC(=O)OCCCCOc1ccc(C(=O)Oc2ccc(OC(=O)c3ccc(OCCCCOC(=O)C=C)cc3)c(C)c2)cc1] 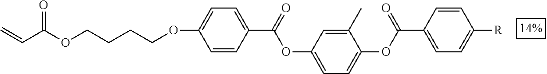 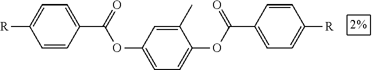 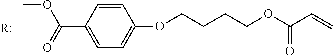
Leveling Agent T-1-

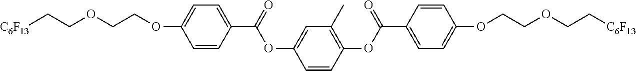

The above-described coating liquid for forming λ/4 layer 1 was applied onto the alignment film 1, the applied coating film was heated to 80° C. using a hot plate, and the coating film was irradiated with ultraviolet rays having a wavelength of 365 om at an irradiation dose of 500 mJ/cm² using a high-pressure mercury lamp in a nitrogen atmosphere, so that the alignment of the liquid crystal compound was immobilized and an optically anisotropic layer (λ/4 layer) was prepared. The obtained layer was set to the λ/4 layer 1. The obtained λ/4 layer 1 had a Δn₈₅₀×thickness d (=Re (850)) of 425 nm. Δn₈₅₀ is the difference in refractive index at a wavelength of 850 nm, and Re (850) is an in-plane retardation at a wavelength of 850 nm.

(Formation of Alignment Film 2)

The coating liquid for forming alignment film 1 was applied onto the λ/4 layer 1 by spin coating. Then, the applied coating film was dried using a hot plate at 60° C. for 60 seconds to form an alignment film 2.

The alignment film 2 was exposed using the exposure device shown in FIG. 10 to form an alignment film 2 having an alignment pattern.

In the exposure device, a laser that emits laser light having a wavelength (325 nm) was used as the laser. The dose of exposure to the interference light was set to 300 mJ/cm². The intersecting angle (intersecting angle α) of the two light rays was set to 26.8°.

(Formation of Liquid Crystal Layer 1)

The following coating liquid for forming liquid crystal layer 1 was prepared as a liquid crystal composition for forming a liquid crystal layer 1 of the liquid crystal diffraction element.

Coating Liquid for Forming Liquid Crystal Layer 1

| | | |
|---|---|---|
| Liquid crystal compound L-1 | 100.00 | parts by mass |
| Polymerization Initiator (Irgacure (registered | 3.00 | parts by mass |
| trademark) 907, manufactured by BASF SE) | | |
| Photosensitizer (KAYACURE DETX-S, | 1.00 | part by mass |
| manufactured by Nippon Kayaku Co., Ltd.) | | |
| Leveling agent T-1 | 0.08 | parts by mass |
| Methyl ethyl ketone | 936.00 | parts by mass |

The liquid crystal layer 1 was formed by multi-layer coating of the coating liquid for forming liquid crystal layer 1 on the alignment film 2. The multi-layer coating refers to repetition of, first, applying the coating liquid for forming liquid crystal layer 1 for a first layer onto the alignment film, heating and cooling the applied coating liquid, and then curing the coating liquid with ultraviolet rays to prepare the liquid crystal immobilized layer, and then of applying the coating liquid for second and subsequent layers to the liquid crystal immobilized layer by overlapping coating, and heating and cooling the applied coating liquid in the same manner, and then curing the coating liquid with ultraviolet rays. The alignment direction of the alignment film 2 is reflected from a lower surface to an upper surface of the liquid crystal layer, even with the liquid crystal layer having the total thickness increased after the formation by the multi-layer coating.

First, in the first layer, the above-described coating liquid for forming liquid crystal layer 1 was applied onto the alignment film 2, the coating film was heated to 80° C. using a hot plate, and the coating film was irradiated with ultraviolet rays having a wavelength of 365 nm at an irradiation dose of 300 mJ/cm² using a high-pressure mercury lamp in a nitrogen atmosphere, so that the alignment of the liquid crystal compound was immobilized.

In the second and subsequent layers, the coating liquid was applied onto the liquid crystal immobilized layer by overlapping coating, was heated and cooled under the same conditions as described above, and then cured with ultraviolet rays, so that the liquid crystal immobilized layer was prepared. In this way, the overlapping coating was repeated until the total thickness reached a desired film thickness, so that the liquid crystal layer 1 was formed. Finally, in the liquid crystal layer 1, the liquid crystal had a Δn₈₅₀×thickness d (=Re (850)) of 425 nm. It was confirmed that the single period of the alignment pattern of the liquid crystal layer 1 was 700 nm. Further, the diffraction angle when light having a wavelength of 850 nm is incident from a direction perpendicular to the liquid crystal layer 1 was 54°. That is, the laser light diffracted by the liquid crystal layer 1 is incident at an angle of 54° with respect to the perpendicularity of the surface of the user U.

(Formation of Alignment Film 3)

The coating liquid for forming alignment film 1 was applied onto the liquid crystal layer 1 by spin coating. Then, the applied coating film was dried in Serco at 60° C. for 60 seconds to form an alignment film 3.

(Exposure of Alignment Film 3)

The alignment film 3 was irradiated with polarized ultraviolet rays (100 mJ/cm², using an ultra-high pressure mercury lamp), so that the alignment film 3 was exposed.

(Formation of Second λ/4 Plate)

The coating liquid for forming λ/4 layer 1 was applied onto the alignment film 3, the applied coating film was heated to 80° C. using a hot plate, and the coating film was irradiated with ultraviolet rays having a wavelength of 365 nm at an irradiation dose of 500 mJ/cm² using a high-pressure mercury lamp in a nitrogen atmosphere, so that the alignment of the liquid crystal compound was immobilized and an optically anisotropic layer (λ/4 layer) that is the second λ/4 plate was prepared. The obtained layer was set to a λ/4 layer 2. The obtained λ/4 layer 2 had a Δn₈₅₀×thickness d (=Re (850)) of 425 nm.

As described above, a laminate including the liquid crystal diffraction element as shown in FIG. 13 was produced.

[Light Source Unit]

As the light source unit, an array in which 10 monochromatic surface emitting lasers (VCSEL) having a wavelength of 850 nm are arranged in a row on the substrate at an interval of 250 μm was used. Further, a light receiving unit (G10899 manufactured by Hamamatsu Photonics K.K.) was disposed at a position 5 mm away from the laser light source in a direction orthogonal to the direction in which the laser light sources are arranged in a row on the substrate. The light receiving unit has a size of φ 0.3 mm.

[Holding Mechanism]

As the frame, a frame that is made of a material of acrylic plastic having an outer size of 14 mm×14 mm and a thickness of 0.5 mm, and that has an opening portion having a size of 10 mm×10 mm in the center was prepared. A pressure-sensitive adhesive gel sheet (Gel tack sheet manufactured by EXSFAL Co., Ltd., a thickness of 2 mm) was cut out into the same shape as the opening surface of the frame and bonded to the frame. Further, the substrate of the light source unit is bonded to the pressure-sensitive adhesive gel sheet on a side opposite to the frame. The laser light sources of the light source unit are disposed in the opening portion.

The laminate including the liquid crystal diffraction element (liquid crystal layer 1) produced above was bonded to the opening surface of the frame on a side opposite to the pressure-sensitive adhesive gel sheet. Upon the bonding, the arrangement direction of the laser light sources and the direction in which the optical axis of the liquid crystal layer 1 rotates in the plane (arrow X1 direction) were disposed so as to be orthogonal to each other.

The liquid crystal diffraction element side was disposed at a position of the radial artery of the wrist of the user U so as to face the user U, the band was wrapped around the wrist from above, and the flow rate measuring device was worn on the wrist of the user U. Upon the wearing, the flow rate measuring device was worn such that the direction in which the optical axis of the liquid crystal layer 1 rotates in the plane (arrow X1 direction) was along the flow direction of the blood vessel V.

[Evaluation]

(Signal Intensity at Rest)

The user U was irradiated with laser light from the laser light source in a state in which the user U wore the flow rate measuring device on the wrist in the above-described manner and sat on the chair with the hand to be measured placed on the desk (hereinafter, also referred to as at rest), and the laser light reflected in the body of the user U was received by the light receiving unit. The signal intensity received by the light receiving unit was read, the magnification was obtained on the basis of Comparative Example 1, which will be described later, and the evaluation was performed according to the following criteria.

A: 3.7 times or more

B: 1.8 times or more and less than 3.7 times

C: More than 1 time and less than 1.8 times

D: 1 time or less

(Signal Intensity at Body Movement)

The signal intensity was read in the same manner as above after the user U performed the bending motion of the arm to be measured while standing (hereinafter, also referred to as at body movement), and the ratio of the signal intensity at body movement to that at rest was calculated and the evaluation was performed according to the following criteria.

A: 1 time or more

B: 0.1 times or more and less than 1 time

C: Less than 0.1 times

### Example 2

A flow rate measuring device was manufactured in the same manner as in Example 1 except that the holding mechanism was not provided, and the above-described evaluation was performed. That is, in Example 2, the laminate including the liquid crystal diffraction element was directly attached to the substrate.

### Example 3

A flow rate measuring device was manufactured in the same manner as in Example 1 except that the intersecting angle α of the two light rays was set to 16.5° in the formation of the alignment film 2, and the above-described evaluation was performed. It was confirmed that the single period of the alignment pattern of the liquid crystal layer 1 (liquid crystal diffraction element) formed on the alignment film 2 was 1.1 μm. The diffraction angle was 30°.

### Example 4

A flow rate measuring device was manufactured in the same manner as in Example 1 except that the intersecting angle α of the two light rays was set to 31.2° in the formation of the alignment film 2, and the above-described evaluation was performed. It was confirmed that the single period of the alignment pattern of the liquid crystal layer 1 (liquid crystal diffraction element) formed on the alignment film 2 was 0.6 μm. The diffraction angle was 700.

### Example 5

A flow rate measuring device was manufactured in the same manner as in Example 1 except that the intersecting angle α of the two light rays was 11.3° in the formation of the alignment film 2, and the above-described evaluation was performed. It was confirmed that the single period of the alignment pattern of the liquid crystal layer 1 (liquid crystal diffraction element) formed on the alignment film 2 was 1.65 μm. The diffraction angle was 20°.

### Example 6

A flow rate measuring device was manufactured in the same manner as in Example 1 except that the intersecting angle α of the two light rays was set to 32.8° in the formation of the alignment film 2, and the above-described evaluation was performed. It was confirmed that the single period of the alignment pattern of the liquid crystal layer 1 (liquid crystal diffraction element) formed on the alignment film 2 was 0.58 μm. The diffraction angle was 80°.

### Example 7

A flow rate measuring device was manufactured in the same manner as in Example 1 except that a prism sheet was used as the light bending member, and the above-described evaluation was performed. That is, the manufacturing method was the same as in Example 1 except that the prism sheet was bonded to the frame instead of the laminate including the liquid crystal diffraction element.

As the prism sheet, LP-40-0.9 (having an inclination angle of 40 degrees, a material of PMMA, and a thickness of 2 mm) manufactured by Nihon Tokushu Kogaku Jushi Co., Ltd. was used. It was confirmed that the prism sheet bent near-infrared laser light having wavelength of 850 nm by 40 degrees.

### Example 8

A flow rate measuring device was manufactured in the same manner as in Example 1 except that a light source unit having a configuration in which a side surface emitting laser and a MEMS mirror were provided was used as the light source unit instead of the light source unit in which laser light sources are arranged in an array and the optical member was provided, and the above-described evaluation was performed.

A monochromatic laser having a wavelength of 850 nm was used as the side surface emitting laser.

The MEMS mirror is a mirror obtained by vapor deposition of gold having high reflectivity in the wavelength range of near-infrared rays. Further, the MEMS mirror is driven by a piezoelectric element. The MEMS mirror was disposed such that the mirror was at 45° with respect to the surface of the substrate.

The optical member was formed in a laminate including a liquid crystal diffraction element. That is, a laminate (see FIG. 26) having a second liquid crystal layer (liquid crystal layer 2) serving as an optical member was produced between the liquid crystal layer 1, which is a liquid crystal diffraction element, and the λ/4 layer 2, which is a second λ/4 plate, as follows.

The forming method from the support to the liquid crystal layer 1 was the same as in Example 1.

(Formation of Alignment Film 4)

The coating liquid for forming alignment film 1 was applied onto the liquid crystal layer 1 by spin coating. Then, the applied coating film was dried in Serco at 60° C. for 60 seconds to form an alignment film 4.

The alignment film 4 was exposed using the exposure device shown in FIG. 25 to form an alignment film 4 having an alignment pattern.

In the exposure device, a laser that emits laser light having a wavelength (325 nm) was used as the laser. The dose of exposure to the interference light was set to 300 mJ/cm². In addition, the single period of the alignment pattern was set so as to be gradually shortened toward the outside.

(Formation of Liquid Crystal Layer 2)

The liquid crystal layer 2, which is the second liquid crystal layer serving as an optical member, was formed by multi-layer coating of the coating liquid for forming liquid crystal layer 1 on the alignment film 4.

Finally, in the liquid crystal layer 2, the liquid crystal had a Δn₈₅₀×thickness d (=Re (850)) of 425 nm. Further, as shown in FIGS. 23 and 24, it was confirmed using the polarization microscope that the liquid crystal alignment pattern was periodic and that the single period of the liquid crystal alignment pattern was gradually shortened from the center toward the outside.

In the single period of the liquid crystal alignment pattern of the liquid crystal layer 2, the rotation period at a position 1.1 mm away from the center was 0.89 μm, the rotation period at a position 2.5 mm away from the center was 0.54 μm, and the rotation period at a position 5.0 mm away from the center was 0.46 μm, and the rotation period was gradually shortened from the center toward the outside.

The alignment film 3 and the second λ/4 plate (λ/4 layer 2) were formed in the same manner as in Example 1 except that the alignment film 3 and the second λ/4 plate were formed on the liquid crystal layer 2, and a laminate including the liquid crystal diffraction element and the optical member was produced.

### Example 9

A flow rate measuring device was manufactured in the same manner as in Example 1 except that the liquid crystal layer 1 was formed by using the following coating liquid for forming liquid crystal layer 1B instead of the coating liquid for forming liquid crystal layer 1 used in Example 1, and the above-described evaluation was performed.

The liquid crystal layer 1 in which the liquid crystal had a Δn₈₅₀×thickness d (=Re (850)) of 425 nm can be obtained even with the coating liquid for forming liquid crystal layer 1B used in which the number of times of multi-layer coating is smaller than that of the coating liquid for funning liquid crystal layer 1, which can be said that the preparation efficiency is high.

Coating Liquid for Forming Liquid Crystal Layer 1B

| | |
|---|---|
| Liquid crystal compound L-2 | 100.00 parts by mass |
| Polymerization Initiator (Irgacure (registered trademark) 907, manufactured by BASF SE) | 3.00 parts by mass |
| Photosensitizer (KAYACURE DETX-S, manufactured by Nippon Kayaku Co., Ltd.) | 1.00 part by mass |
| Leveling agent T-1 | 0.08 parts by mass |
| Tetrahydrofuran | 936.00 parts by mass |

Liquid Crystal Compound L-2-

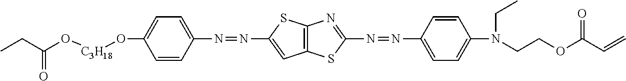

### Example 10

A flow rate measuring device was manufactured in the same manner as in Example 1 except that the intersecting angle α of the two light rays was set to 21.20 in the formation of the alignment film 2, and the above-described evaluation was performed. The diffraction angle was 40°.

### Comparative Example 1

A flow rate measuring device was manufactured in the same manner as in Example 1 except that the laminate including the liquid crystal diffraction element and a holding mechanism (the frame and the pressure-sensitive adhesive gel sheet) were not provided, and the above-described evaluation was performed. That is, in Comparative Example 1, the substrate on which the laser light source and the light receiving unit were mounted was brought into direct contact with the wrist of the user U, the band was wrapped around the wrist, and the flow rate measuring device was worn on the wrist of the user U.

### Comparative Example 2

A flow rate measuring device was manufactured in the same manner as in Example 8 except that the laminate including the liquid crystal diffraction element and a holding mechanism (the frame and the pressure-sensitive adhesive gel sheet) were not provided, and the above-described evaluation was performed.

TABLE 1

| | Light | | | Holding | Evaluation | |
|---|---|---|---|---|---|---|
| | source unit | Light bending member | | mechanism | Signal intensity | |
| | Configu- | | Bending | Provided/ | | At body |
| | ration | Type | angle ° | Not provided | At rest | movement |
| Example 1 | Array | Liquid crystal | 54 | Provided | A | A |
| | | diffraction element | | | | |
| Example 2 | Array | Liquid crystal | 54 | Not provided | A | B |
| | | diffraction element | | | | |
| Example 3 | Array | Liquid crystal | 30 | Provided | B | A |
| | | diffraction element | | | | |
| Example 4 | Array | Liquid crystal | 70 | Provided | B | A |
| | | diffraction element | | | | |
| Example 5 | Array | Liquid crystal | 20 | Provided | C | A |
| | | diffraction element | | | | |
| Example 6 | Array | Liquid crystal | 80 | Provided | C | A |
| | | diffraction element | | | | |
| Example 7 | Array | Prism sheet | 40 | Provided | B | A |
| Example 8 | MEMS | Liquid crystal | 54 | Provided | A | A |
| | mirror | diffraction element | | | | |
| Example 9 | Array | Liquid crystal | 54 | Provided | A | A |
| | | diffraction element | | | | |
| Example 10 | Array | Liquid crystal | 40 | Provided | A | A |
| | | diffraction element | | | | |
| Comparative | Array | None | 0 | Not provided | D | C |
| Example 1 | | | | | | |
| Comparative | MEMS | None | 0 | Not provided | D | C |
| Example 2 | mirror | | | | | |

From Table 1, it is seen that the signal intensity in Examples of the present invention having the light bending member is higher than that in Comparative Example.

Further, from the comparison of Examples 1, 3 to 6, it is seen that the angle of the laser light bent by the light bending member with respect to the perpendicular line to the surface of the user U is preferable 30° to 70°.

Further, from the comparison between Examples 1 and 2, it is seen that it is preferable to have the holding mechanism.

Further, from the comparison Example 10 and 7, it is seen that it is preferable to use the liquid crystal diffraction element as the light bending member.

### EXPLANATION OF REFERENCES

10: flow rate measuring device
12: substrate
14, 14a to 14c: laser light source
16: light receiving unit
18: light bending member
20: light condensing member
21: holding mechanism
22: elastic member
24: frame
25, 31, 32, 37: alignment film
26: optical member
27, 36: liquid crystal layer
28: alignment mechanism (MEMS mirror)
28a: mirror
30: support
33: first λ/4 plate
35: liquid crystal diffraction element
38: second λ/4 plate
40: liquid crystal compound
40A: optical axis
60, 80: exposure device
62, 82: laser
64, 84: light source
65: λ/2 plate
68, 86, 94: polarizing beam splitter
70A, 70B, 90A, 90B: mirror
72A, 72B, 96: λ/4 plate
92: lens
100: band
102: display
D1: diffraction element
D2: prism sheet
D3: prism
D4: mirror
X1: one direction
L₁, L₄: incidence light
L₂, L₅: transmitted light
Q1, Q2: absolute phase
E1, E2: equiphase plane
A: single period
M: laser light
MA, MB: light ray
MP: P-polarized light
MS: S-polarized light
P_{O}: linearly polarized light
P_{R}: right circularly polarized light
P_{L}: left circularly polarized light
U: user
V: blood vessel
V₁ to V₃: blood vessel position

What is claimed is:

1. A flow rate measuring device that detects a flow velocity of a fluid flowing through an object by a Doppler effect of light, the flow rate measuring device comprising:
a light source unit that emits laser light to the object;
a light receiving unit that receives the laser light scattered by the object; and
a light bending member that bends the laser light emitted from the light source unit and causes the laser light to be inclined with respect to a surface of the object and incident on the surface.

2. The flow rate measuring device according to claim 1, further comprising:
a holding mechanism that keeps a distance between the light bending member and the object constant.

3. The flow rate measuring device according to claim 1,
wherein the laser light bent by the light bending member is incident on the object at an angle of 30° to 70° with respect to a perpendicular line to the surface of the object.

4. The flow rate measuring device according to claim 1,
wherein the light bending member includes at least one of a prism sheet, a lens sheet, or a liquid crystal diffraction element.

5. The flow rate measuring device according to claim 1,
wherein the light source unit includes a substrate and a plurality of laser oscillating elements provided on the substrate.

6. The flow rate measuring device according to claim 1,
wherein the light source unit has
a laser oscillating element,
an alignment mechanism that performs scanning with laser light emitted from the laser oscillating element, and
an optical member that makes an incidence angle of the laser light with which the scanning is performed by the alignment mechanism, on the light bending member constant.

7. The flow rate measuring device according to claim 6,
wherein the optical member includes any one of a liquid crystal lens or a gradient index lens.

8. The flow rate measuring device according to claim 2,
wherein the laser light bent by the light bending member is incident on the object at an angle of 30° to 70° with respect to a perpendicular line to the surface of the object.

9. The flow rate measuring device according to claim 2,
wherein the light bending member includes at least one of a prism sheet, a lens sheet, or a liquid crystal diffraction element.

10. The flow rate measuring device according to claim 2,
wherein the light source unit includes a substrate and a plurality of laser oscillating elements provided on the substrate.

11. The flow rate measuring device according to claim 2,
wherein the light source unit has
a laser oscillating element,
an alignment mechanism that performs scanning with laser light emitted from the laser oscillating element, and
an optical member that makes an incidence angle of the laser light with which the scanning is performed by the alignment mechanism, on the light bending member constant.

12. The flow rate measuring device according to claim 11,
wherein the optical member includes any one of a liquid crystal lens or a gradient index lens.

13. The flow rate measuring device according to claim 3,
wherein the light bending member includes at least one of a prism sheet, a lens sheet, or a liquid crystal diffraction element.

14. The flow rate measuring device according to claim 3,
wherein the light source unit includes a substrate and a plurality of laser oscillating elements provided on the substrate.

15. The flow rate measuring device according to claim 3,
wherein the light source unit has
a laser oscillating element,
an alignment mechanism that performs scanning with laser light emitted from the laser oscillating element, and
an optical member that makes an incidence angle of the laser light with which the scanning is performed by the alignment mechanism, on the light bending member constant.

16. The flow rate measuring device according to claim 15,
wherein the optical member includes any one of a liquid crystal lens or a gradient index lens.

17. The flow rate measuring device according to claim 4,
wherein the light source unit includes a substrate and a plurality of laser oscillating elements provided on the substrate.

18. The flow rate measuring device according to claim 4,
wherein the light source unit has
a laser oscillating element,
an alignment mechanism that performs scanning with laser light emitted from the laser oscillating element, and
an optical member that makes an incidence angle of the laser light with which the scanning is performed by the alignment mechanism, on the light bending member constant.

19. The flow rate measuring device according to claim 18,
wherein the optical member includes any one of a liquid crystal lens or a gradient index lens.
